(19) United States
(12) Patent Application Publication
(10) Pub. No.: US 2024/0255510 A1
(43) Pub. Date: Aug. 1, 2024

(54) SALIVARY METABOLITES ARE NON-INVASIVE BIOMARKERS OF HCC

(71) Applicant: THE CLEVELAND CLINIC FOUNDATION, Cleveland, OH (US)

(72) Inventors: Daniel ROTROFF, Pepper Pike, OH (US); Federico AUCEJO, Shaker Heights, OH (US); Courtney HERSHBERGER, Brooklyn Heights, OH (US)

(21) Appl. No.: 18/290,150

(22) PCT Filed: May 10, 2022
(86) PCT No.: PCT/US2022/028612

§ 371 Date: Nov. 9, 2023

Related U.S. Application Data
(60) Provisional application No. 63/186,479, filed on May 10, 2021.

(51) Int. Cl. G01N 33/574

(52) U.S. Cl. CPC G01N 33/57438; G01N 33/57488

(57) ABSTRACT

Metabolites detectable in saliva are useful for indicating disease pathology or a breakdown in liver function. The relative abundance of particular combinations of salivary metabolites derived from machine-learning serve as non-invasive biomarkers of HCC. Combinatorial patterns of salivary metabolites can distinguish healthy individuals from those having cirrhosis and/or hepatocellular carcinoma (HCC). Accordingly, the disclosure provides methods for evaluating the HCC status of a subject and providing treatment appropriate for the HCC status of the subject.

### CROSS-REFERENCE TO RELATED APPLICATIONS

This application claims the benefit of priority to U.S. Provisional Application No. 63/186,479, filed May 10, 2021; the contents of which are hereby incorporated by reference in their entirety and for all purposes.

### STATEMENT REGARDING FEDERALLY SPONSORED RESEARCH OR DEVELOPMENT

This invention was made in part with U.S. Government support under Grant Nos. R01-DK120679; P50-AA024333; and P01-HL147823 awarded by the U.S. National Institutes of Health. The U.S. Government has certain rights to this invention.

### FIELD OF THE DISCLOSURE

The present disclosure relates generally to the use of machine learning to detect salivary metabolite signatures useful as diagnostic biomarkers for hepatocellular carcinoma (HCC).

### BACKGROUND OF THE DISCLOSURE

Liver cancer is the most rapidly increasing cancer in the United States and is estimated to have resulted in 31,780 deaths in 2019 (Cancer Facts & FIGS. 2019. American Cancer Society: Atlanta, GA, USA). Hepatocellular carcinoma (HCC) which is the most common liver cancer, accounts for 80% of all primary liver cancers, and the global incidence of HCC is expected to increase to 78 million by 2030 (Petrick J L, et al. (2016) Journal of Clinical Oncology 34:1787). A majority of patients that develop HCC have preexisting cirrhosis, and HCC is leading cause of death among individuals with cirrhosis. Cirrhosis can develop after infection with hepatitis B or hepatitis C, heavy alcohol consumption, or in individuals with chronic liver diseases such as nonalcoholic fatty liver disease (NAFLD) and nonalcoholic steatohepatitis (NASH).

The prevalence of liver cancers has been steadily rising since the 1970s due to hepatitis C infections and increases in obesity resulting in chronic liver diseases. HCC is one of the most aggressive, common neoplasias in the world, characterized by an often unfavorable course. The main therapies with curative potential for cases of hepatocellular carcinoma involve surgical resection or a liver transplant. However, the low postoperative survival rate (30-40% after five years) and frequent post-surgery reappearance of metastasis in patients undergoing a surgical resection treatment considerably complicate the clinical approach toward hepatocellular carcinoma. This limit is further exacerbated by the reduced possibility of surgical treatment, which is in fact restricted to only a small percentage of patients (around 20% of patients with hepatocellular carcinoma), in particular those patients found to have small lesions and relatively normal hepatic parameters.

Early detection of HCC has been shown to improve reception of curative therapy and overall survival (Singal A G, et al. PLoS medicine. 2014; 11: e1001624). Unfortunately, however, current HCC serum biomarkers, such as alpha fetoprotein (AFP) and ultrasound, lack prognostic and diagnostic value and result in many false negative diagnoses (Daniele B, et al. (2004) Gastroenterology 127: S108-S112; Ayuso C, et al. (2018) European journal of radiology. 101: 72-81). Thus, reliable early detection remains elusive.

Therefore, additional methods and biomarkers that inform the surveillance of patients at risk for HCC could help to prevent false negative diagnoses and enable curative treatment options prior to the onset of advanced disease. As such, there remains a need for improved methods for detection HCC, and for distinguishing patients having HCC from those having cirrhosis. Fortunately, the following disclosure provides for this and other needs.

### SUMMARY OF THE DISCLOSURE

The present disclosure provides method of using a predictive Random Forest machine-learning algorithm and metabolites in a patient's saliva to discern healthy individuals from those with hepatocellular carcinoma (HCC) or cirrhosis. Thus, the disclosure provides salivary metabolite signatures that are highly sensitive and specific non-invasive biomarkers of HCC. The salivary metabolite signatures disclosed herein reflect the differential abundance of particular metabolites in the saliva of patients with HCC compared to the saliva of patients with cirrhosis and are able to distinguish patients having HCC from those having cirrhosis. Therefore, provided herein are methods, kits, and compositions related to diagnosing and treating HCC in a subject.

In accordance with one aspect of the disclosure, there is provided a method for diagnosing or prognosticating HCC in a subject, or for assessing the risk of developing HCC, or for monitoring the effectiveness of a therapy for HCC, comprising: determining, in an isolated sample of saliva, the level of abundance of at least one salivary metabolite selected from the group comprising or consisting of octadecanol, acetophenone, lauric acid, 1-monopalmitin, dodecanol, salicylaldehyde, glycyl-proline, 1-monosterin, creatinine, glutamine, serine, and 4-hydroxybutyric acid, and combinations thereof, and determining whether the at least one salivary metabolite is differentially abundant compared to a reference sample, wherein differential abundance of the at least one salivary metabolite is an increase or a decrease, in order to determine the HCC status of the subject, and treating the subject diagnosed with HCC or having elevated risk of HCC or as needing more effective treatment for HCC, with a compound or other therapy to improve the HCC status of the subject

In some embodiments, methods for diagnosing or prognosticating HCC in a subject, or for assessing the risk of developing HCC, or for monitoring the effectiveness of a therapy for HCC, are provided comprising: determining, in an isolated sample of saliva, the level of abundance of at least one salivary metabolite selected from the group comprising or consisting of acetophenone, octadecanol, 1-monopalmitin, 1-monostearin, lauric acid, 3-hydroxybutyric acid, and combinations thereof, and determining whether the at least one salivary metabolite is differentially abundant compared to a reference sample, wherein differential abundance of the at least one salivary metabolite is an increase or a decrease, in order to determine the HCC status of the subject, and treating the subject diagnosed with HCC or having elevated risk of HCC or as needing more effective treatment for HCC, with a compound or other therapy to improve the HCC status of the subject.

In one embodiment, the reference sample is from a subject who does not have HCC. In one embodiment, the reference sample is from a subject who has cirrhosis. In one embodiment, the level of abundance of acetophenone in the subject is decreased as compared to the reference sample, and the subject is diagnosed as having HCC. In one embodiment, the reference sample is from a healthy subject. In one embodiment, the level of abundance of acetophenone in the subject is decreased as compared to the reference sample and the subject is diagnosed as having cirrhosis or HCC. In one embodiment, the reference sample is from a subject who has cirrhosis, the level of abundance of acetophenone in the subject is decreased as compared to the reference sample, and the subject is diagnosed as having HCC. In one embodiment, the level of abundance of octadecanol is decreased as compared to the reference sample and the subject is diagnosed as having cirrhosis or HCC. In one embodiment, the reference sample is from a subject who has cirrhosis and the subject is diagnosed as having HCC. In one embodiment, the method has sensitivity of at least 88% and specificity of at least 94%.

In another aspect the disclosure provides a method for differentiating a subject having hepatocellular carcinoma (HCC) from a subject having liver cirrhosis, the method comprising the step of determining, in an isolated sample of saliva, the level of abundance of at least one salivary metabolite selected from the group comprising or consisting of octadecanol, acetophenone, lauric acid, 1-monopalmitin, dodecanol, salicylaldehyde, glycyl-proline, 1-monosterin, creatinine, glutamine, serine, and 4-hydroxybutyric acid, and combinations thereof, and determining whether the at least one salivary metabolite is differentially abundant compared to a reference sample from a subject having cirrhosis, wherein differential abundance of the at least one salivary metabolite is an increase or decrease, and wherein differential abundance of the at least one salivary metabolite indicates the subject has HCC.

In some embodiments, methods for differentiating a subject having hepatocellular carcinoma (HCC) from a subject having liver cirrhosis are provided comprising the step of determining, in an isolated sample of saliva, the level of abundance of at least one salivary metabolite selected from the group comprising or consisting of acetophenone, octadecanol, 1-monopalmitin, 1-monostearin, lauric acid, 3-hydroxybutyric acid, and combinations thereof, and determining whether the at least one salivary metabolite is differentially abundant compared to a reference sample from a subject having cirrhosis, wherein differential abundance of the at least one salivary metabolite is an increase or decrease, and wherein differential abundance of the at least one salivary metabolite indicates the subject has HCC. In one embodiment, the at least one salivary metabolite is acetophenone. In one embodiment, the at least one salivary metabolite is octadecanol. In one embodiment, the at least one salivary metabolite is lauric acid. In one embodiment, the at least one salivary metabolite is 3-hydroxybutyric acid.

In one embodiment, the at least one salivary metabolite is at least four salivary metabolites. In one embodiment, when the four salivary metabolites include acetophenone, and/or octadecanol, the four salivary metabolites do not include lauric acid, and/or 3-hydroxybutyric acid. Thus, in one embodiment, the at least four salivary metabolites are: acetophenone, octadecanol, 1-monopalmitin, 1-monostearin. In another embodiment, the at least four salivary metabolites are: lauric acid, 3-hydroxybutyric acid, 1-monopalmitin, and 1-monostearin. In some embodiments the method further comprises the additional step of treating the subject diagnosed with HCC with a compound or other therapy.

In another aspect the disclosure provides a method for discovering salivary biomarkers of HCC in saliva from a subject having HCC, die method comprising: (a) obtaining or having obtained a saliva sample from the subject, and a saliva sample from a subject not having liver disease, and (b) detecting metabolites that are differentially abundant in the subject having HCC compared to the subject not having ICC, wherein the differentially abundant metabolites have a high predictive value for detection of HCC, and wherein the detection step comprises machine learning utilizing Random Forest and least absolute shrinkage and selection operator (LASSO) and cross-validation, and thereby identifying differentially abundant salivary metabolites that are biomarkers of HCC.

In one embodiment, the differentially abundant salivary metabolites are selected from the group comprising or consisting of: 1-kestose, 3-(4-hydroxyphenyl)propionic acid, Proline, Propane-1,3-diol, Putrescine, Pyruvic acid, Salicylaldehyde, Serine, Sophorose, Sorbitol, Spermidine, Squalene, 3-aminoisobutyric acid, Stearic acid, Succinic acid, Sucrose, Threitol, Threonic acid, Threonine, Thymine, Tocopherol alpha-, Tryptophan, Tyrosine, 3-hydroxybutyric acid, Uracil, Urea, Uric acid, Valine, Xanthine, Xylitol, 3-phenyllactic acid, 3-phosphoglycerate, 4-aminobutyric acid, 4-hydroxybutyric acid, 4-hydroxyphenylacetic acid, 4716, 5-aminovaleric acid, 1-monopalmitin, Acetophenone, Adenine, Adenosine-5-monophosphate, Alanine, Alanine-alanine, Aminomalonate, Arabitol, Arachidic acid, Asparagine, Benzoic acid, 1-monostearin, Beta-glycerolphosphate, Butane-2,3-diol, Butylamine, Butyrolactam, Cellobiose, Cerotinic acid, Cholesterol, Citramalic acid, Citric acid, Citrulline, 1,5-anhydroglucitol, Creatinine, Cysteine, Cystine, D-erythro-sphingosine, Dodecanol, Fructose, Fructose-6-phosphate, Fucose, Fumaric acid, Galactinol, 2-aminobutyric acid, Glucose, Glucose-1-phosphate, Glucose-6-phosphate, Glutamic acid, Glutamine, Glutaric acid, Glyceric acid, Glycerol, Glycerol-alpha-phosphate, Glycine, 2-deoxypentitol, Glycolic acid, Glycyl tyrosine, Glycyl-proline, Guanidinosuccinate, Guanine, Heptadecanoic acid, Homoserine, Indole-3-acetate, Inulotriose, Isoleucine, 2-hydroxyglutaric acid, isomaltose, Isothreonic acid, Lactic acid, Lauric acid, Leucine, Levoglucosan, Lysine, Maleic acid, Maleimide, Malonic acid, 2-ketoisocaproic acid, Maltose, Maltotriose, Melezitose, Methionine, Methionine sulfoxide, Myo-inositol, Myristic acid, N-carbamoylaspartate, Nicotinic acid, Octadecanol, 2,5-dihydroxypyrazine, Oleamide, Oleic acid, Ornithine, Oxoproline, Palmitic acid, Phenylacetic acid, Phenylalanine, Phosphate, Phosphoethanolamine, and Piperidone.

In some embodiments, salivary metabolic biomarkers of HCC are selected from the group comprising or consisting of octadecanol, acetophenone, lauric acid, 1-monopalmitin, dodecanol, salicylaldehyde, glycyl-proline, 1-monosterin, creatinine, glutamine, serine, and 4-hydroxybutyric acid, and combinations thereof. In one embodiment, salivary metabolic biomarkers of HCC are selected from the group comprising or consisting of acetophenone, octadecanol, 1-monopalmitin, 1-monostearin, lauric acid, 3-hydroxybutyric acid, and combinations thereof.

Other features objects and advantages will be apparent from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1. Principal component analysis (PCA) revealed variation in the metabolite relative abundance due to experimental batch as evidenced by the separation of these labeled technical replicates. Mean centering and scaling by the metabolite standard deviations were effective for neutralizing differences due to batch.

FIG. 2. A random forest model (RF125) including all detected metabolites was used to classify subjects by disease status. Hyperparameter optimization was performed using a grid search to identify the optimal number of trees (ntree), and 150 was chosen as the optimal ntree value based on the mean misclassification, sensitivity, and specificity across the LOOCV iterations.

FIG. 3. Workflow diagram for data collection, processing, analysis and generation of predictive models for disease state classification using metabolite relative abundance.

FIG. 4. Eight metabolites differ between patient cohorts. a) Volcano plot depicting false discovery rate (FDR) and Log2 Fold Change (Log2 FC) derived for all metabolites in pair-wise comparisons of disease status, adjusted for differences in age and sex. Metabolites with an FDR P<0.2 (dotted red line) are highlighted. b) Box plots displaying distribution of relative abundance stratified by disease status for significantly differing metabolites in at least one comparison (FDR P<0.2, adjusted for age and sex).

FIG. 5. Random Forest™ model predicts disease status from metabolite abundance. a) An iterative random forest (iRF) approach was used, whereby RF models were generated after iteratively removing the metabolite with the lowest mean Gini score within a leave-one-out cross-validation (LOOCV) framework. The range (min, mean, max) of OOB error across the models is displayed. The model including 125 metabolites is shown in red (RF125), the model including 12 metabolites is shown in blue (iRF12) and the model including 4 metabolites is shown in red (iRF4). b) The range of Gini scores (minimum, mean, maximum) across all metabolites in model RF125. Red coloring indicates the selected metabolites for the iRF models. c) The range of Gini scores of metabolites included in the iRF8. d) The range of Gini scores of metabolites included in iRF4.

FIG. 6. Classification of disease status predicted by decision tree model. a) A decision tree model based on selected metabolites from the iterative random forest (iRF12) approach optimized with a classification accuracy of 86%. Colored squares indicate the disease status of each individual by disease status at each branch of the decision tree. b) Comparison of accuracy metrics from RF with all metabolites (RF125), iRFs with selected metabolites (iRF12, iRF4), and the decision tree models (DT) using leave-one-out cross-validation. Thus, utilizing combinations of multiple metabolites to provide a salivary metabolite signature, it is possible to discriminate between healthy individuals, those with cirrhosis, and those with HCC with high accuracy.

FIG. 7. 4-hydroxybutyric acid is not significantly associated with sex (P>0.05). a) Proportion of males and females after splitting based on relative abundance of 4-hydroxybutyric acid. b) Relative abundance of 4-hydroxybutyric acid stratified by sex across the entire cohort. c) Accuracy metrics of three classification models generated after exclusion of 4-hydroxybutyric acid using leave-one-out cross-validation (LOOCV).

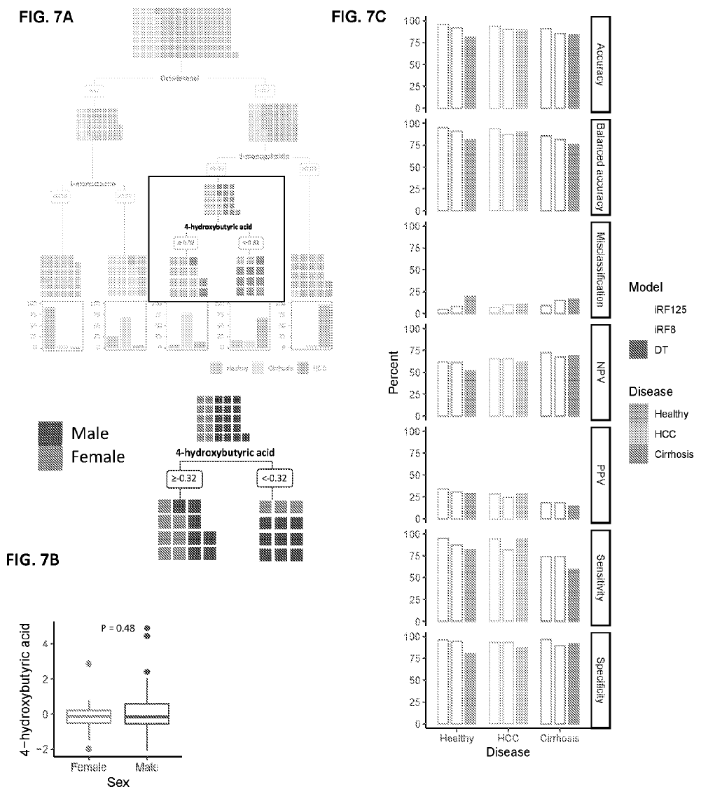

FIG. 8. A decision tree model based on selected metabolites from iterative random forest iRF12, trained to discriminate between diagnoses. Colored squares indicate the BCLC stage of individuals with HCC. Barplots indicate the proportion of subjects who are healthy, have cirrhosis or HCC in the terminal decision tree leaves. No discernable patterns were detected based on BCLC stage, and a single patient with HCC with BCLC stage 0 and 13/15 patients with BCLC stage A were classified correctly, indicating that the model is capable of detecting individuals with early stage or minimal disease.

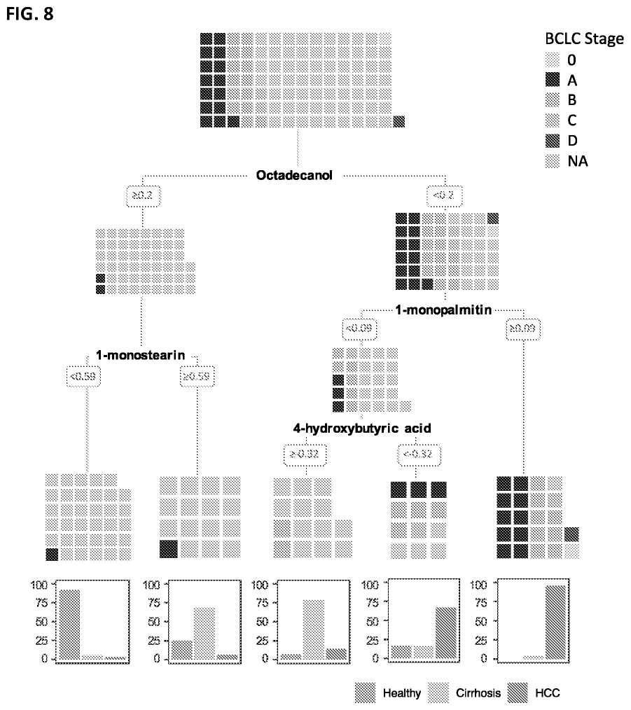

FIG. 9. A decision tree model based on selected metabolites from iterative random forest iRF12, trained to discriminate between diagnoses. Colored squares indicate the Child-Pugh Class of individuals with cirrhosis and HCC. Barplots indicate the proportion of subjects who are healthy, have cirrhosis or HCC in the terminal decision tree leaves. No discernable patterns were detected based on Child-Pugh class, indicating that the model is specific for HCC even when the patient has a high Child-Pugh class (i.e., class C).

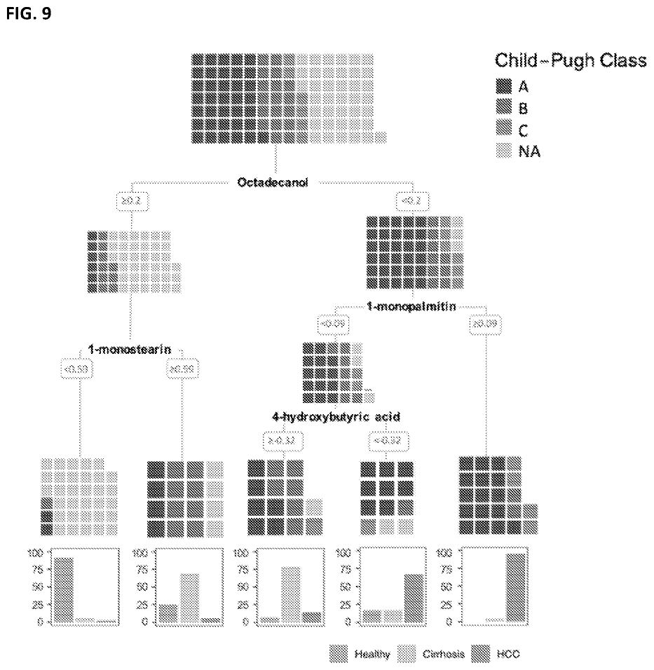

### DETAILED DESCRIPTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, where indicated, the term “about” indicates the designated value±one standard deviation of that value.

The term “combinations thereof” includes every possible combination of elements to which the term refers.

The term “subject” or “patient” as used herein, refers to an individual or mammal having a disease or at elevated risk of having a disease (e.g., having or at elevated risk of having HCC). The “subject” may be diagnosed to be affected by e.g., HCC, or may be diagnosed e.g., to have liver cirrhosis. Similarly, a “subject” may further be diagnosed to be at elevated risk of developing HCC e.g., may have liver cirrhosis. The subject may be any mammal, including both a human and another mammal, e.g. an animal such as a rabbit, mouse, rat, or monkey. Human subjects are preferred.

The term “liver disease” or “hepatic disease” as used herein refers to any liver dysfunction or disturbance that causes the liver to fail to perform its full spectrum functions, thus resulting in illness in the present or in the future. Thus, the term “liver disease” includes those subjects who may be at elevated risk liver disease e.g., at elevated risk of cirrhosis, or elevated risk of HCC. Exemplary liver diseases include, but are not limited to cirrhosis and hepatocellular carcinoma (HCC).

The term “metabolites” as used herein, refers to biologically derived molecules that are the intermediates or end products of metabolism. Thus, “metabolites” are small molecule products of biological processes. “metabolites” are readily be measured using techniques such as e.g., mass spectrometry or nuclear magnetic resonance (NMP). “Salivary metabolites” as used herein refers metabolic products found in the saliva.

Exemplary salivary metabolites include, e.g., “octadecanol” which may be equivalently referred to as “stearyl alcohol,” “octadecan-1-ol,” “1-octadecanol,” “octadecanol,” or “octadecyl alcohol;” “acetophenone,” which may be equivalently referred to as “methyl phenyl ketone,” “1-phenylethanone,” “methyl phenyl ketone,” or “phenyl methyl ketone;” “1-monopalmitin” which may be equivalently referred to as “1-monopalmitate glycerol,” “2,3-dihydroxypropyl hexadecanoate,” “palmitic acid alpha-monoglyceride,” “1-palmitoyl-rac-glycerol,” “monopalmitin,” or “glyceryl palmitate;” “1-monostearin” which may be equivalently referred to as “1-monostearate-glycerol,” “2,3-dihydroxypropyl octadecanoate,” “stearic acid glyceryl ester,” “glyceryl monostearate,” “monostearin,” or “glyercol monostearate.”

The term “saliva” as used herein refers to whole-mouth saliva (WMS), the fluid produced upon spitting or drooling.

The term “salivary metabolite signature” or “metabolite signature” as used herein refers to a characteristic pattern of relative abundance of multiple metabolites present in saliva. In some embodiments, the characteristic pattern of relative abundance reflects the “liver disease status” of a subject.

The term “reference level,” “reference sample,” “control level,” “control sample,” or grammatically equivalent expressions are used interchangeably herein to refer to a reference sample to which a test sample from a subject is compared. The nature of the reference sample depends on the particular diagnosis to be made. For example, to determine if a subject having liver cirrhosis also has HCC or is at elevated risk of developing HCC, the “reference level,” or “reference sample” may be a salivary metabolite signature from a subject known to have cirrhosis, but not HCC. Alternatively, a “reference sample” may be a salivary metabolite signature from a healthy subject without HCC or any cancer related diseases. Appropriate controls are readily chosen by a person having ordinary skill in the art.

The term “differentially abundant” or “differential abundance” as used herein refers to metabolites which differ in relative abundance between a test sample and a reference sample or control, for example which differ in abundance between a healthy patient and a patient having HCC and/or a patient having cirrhosis. Metabolites are differentially abundant when their level of abundance are either higher or lower than abundance in a reference sample or control.

The term “accuracy” as used herein, has the meaning commonly understood in the art (see e.g., Fawcett, Tom (2006) Pattern Recognition Letters. 27 (8): 861-874) and refers to the degree of closeness of measurements of a quantity to that quantity's true value, and is calculated as the sum of true positives plus true negatives divided by the sum of all positives and all negatives.

The term “sensitivity” as used herein has the meaning commonly understood in the art (see e.g., Fawcett, (2006) supra). “Sensitivity” is a statistical measure of how well a binary classification test correctly identifies a condition, and refers to the ability of the analytical method or algorithm to truly determine the individuals that have the disease. Thus, sensitivity is a measure of how well a test can identify true positives. As known in the art (Yerushalmy, J. (1947) Public Health Reports. 62 (2): 1432-39; Fawcett, Tom (2006) Pattern Recognition Letters. 27 (8): 861-874; Powers, David M W (2011) Journal of Machine Learning Technologies. 2 (1): 37-63), sensitivity measures the proportion of positives that are correctly identified (e.g. the proportion of those who have HCC who are correctly identified as having the condition). Thus, Sensitivity=True Positive/(True Positive+False Negative)×100%.

The term “specificity” as used herein has the meaning commonly understood in the art (see e.g., Fawcett, (2006) supra). “Specificity” is a statistical measure of how well a binary classification test correctly identifies a condition, for example how frequently it correctly classifies a subject having HCC or at elevated risk of developing HCC. “Specificity” measures the proportion of negatives that are correctly identified (e.g. the proportion of those who do not have HCC who are correctly identified as not having HCC). Thus, Specificity=True Negative/(False Positive+True Negative)×100% or 1-false positive rate.

A discussion of “sensitivity” and “specificity” as known in the art can be found, for example, on the world wide web at en.wikipedia.org/wiki/Sensitivity_and_specificity.

The term “predictive value” or “positive predictive value” as used herein refers to the ratio of true positives out of all identified positives.

The term “Receiver operating characteristic (ROC) curves” refers to a graphical measure of sensitivity (y-axis) vs. 1—specificity (x-axis) for a clinical test, which is known in the art (see e.g., Fawcett, (2006) supra). A measure of the accuracy of a clinical test is the area under the ROC curve value (AUC value). If this area is equal to 1.0 then this test is 100% accurate because both the sensitivity and specificity are 1.0 so there are no false positives and no false negatives. On the other hand a test that cannot discriminate is the diagonal line from 0,0 to 1,1. The ROC area for this line is 0.5. ROC curve areas (AUC-values) are typically between 0.5 and 1.0. Thus, an AUC-value close to 1 (e.g. 0.95) represents a clinical test as that has high sensitivity and specificity and accuracy.

The term “biomarker” as used herein, refers to a characteristic that can be objectively measured and evaluated as an indicator of normal and disease processes or pharmacological responses. A “biomarker” is a parameter that can be used to measure the onset or the progress of disease or the effects of treatment. The parameter can be chemical, physical or biological.

As used herein, “treating” or “treatment” of any disease or disorder refers, in certain embodiments, to ameliorating a disease or disorder that exists in a subject. “Treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder. For example, in an exemplary embodiment, the phrase “treating cancer” refers to inhibition of cancer cell proliferation, inhibition of cancer spread (metastasis), inhibition of tumor growth, reduction of cancer cell number or tumor growth, decrease in the malignant grade of a cancer (e.g., increased differentiation), or improved cancer-related symptoms. Further, as used herein, “treatment” includes preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer.

As used herein, the term “therapeutically effective amount” or “effective amount” refers to an amount of the subject compositions that when administered to a subject is effective to treat a disease or disorder. For example, in an exemplary embodiment, the phrase “effective amount” is used interchangeably with “therapeutically effective amount” or “therapeutically effective dose” and the like, and means an amount of a therapeutic agent that is effective for treating cancer. Effective amounts of the compositions provided herein may vary according to factors such as the disease state, age, sex, weight of the animal.

### I. Introduction

Current approaches used to screen patients for HCC lack sensitivity and accuracy, resulting in too any false negative diagnoses. Furthermore, in many cases it is not possible to distinguish patients who are experiencing HCC and cirrhosis from those who experience cirrhosis only. Accordingly, there is a significant need for more sensitive screening tools to detect HCC that would allow for early diagnosis, and additionally that can distinguish whether or not a patient is healthy, experiencing cirrhosis alone, or suffering from cirrhosis and HCC. Ideally, such screening tools would also be non-invasive, easy to use, and cost-effective so that they are widely accessible, thus improving patient outcome.

Accordingly, the present disclosure provides a method of using a predictive Random Forest machine-learning algorithm and particular metabolites (octadecanol, acteophenone, 1-monopalmitin, 1-monostearin) in a patient's saliva to discern healthy individuals from those with hepatocellular carcinoma (HCC) or cirrhosis.

Accordingly, the present disclosure provides a method of using a predictive decision tree classification algorithm and particular metabolites (octadecanol, 4-hydroxybutyric acid, 1-monopalmitin, 1-monostearin) in a patient's saliva to discern healthy individuals from those with hepatocellular carcinoma (HCC) or cirrhosis.

### II. General Methods

A patient suspected of having HCC can be identified by any method known in the art. A patient suspected of having HCC can be identified by behavioral or experiential circumstances or by physical or clinical symptoms. For example, the risk of HCC is typically higher in people with long-term liver diseases. Thus, patients experiencing hepatitis B or hepatitis C may have or be suspected of having HCC. HCC is also more common in people who drink large amounts of alcohol, who take certain drugs such as e.g., anabolic steroids, who have too much iron stored in the liver, who experience exposure to aflatoxins, and/or individuals who have an accumulation of fat in the liver such as individuals who have obesity or who have diabetes.

Typically, early stages of HCC do not present any symptoms. Thus, determining whether a patient is suspected of having HCC may be made by a physician based on patient history. However, later stages of HCC often exhibit symptoms such as e.g., upper abdominal pain, weight loss, jaundice, fluid in the abdomen, and/or liver failure.

This disclosure utilizes routine techniques in the field of recombinant genetics. Basic texts disclosing the general methods and terms in molecular biology and genetics include e.g., Sambrook et al., Molecular Cloning, a Laboratory Manual, Cold Spring Harbor Press 4th edition (Cold Spring Harbor, N.Y. 2012); Current Protocols in Molecular Biology Volumes 1-3, John Wiley & Sons, Inc. (1994-1998). This disclosure also utilizes routine techniques in the field of biochemistry. Basic texts disclosing the general methods and terms in biochemistry include e.g., Lehninger Principles of Biochemistry sixth edition, David L. Nelson and Michael M. Cox eds. W. H. Freeman (2012).

This disclosure also utilizes routine methods in the fields of statistics and machine learning. Basic texts disclosing the general methods and terms statistics and machine learning include e.g., Fawcett, Tom (2006) Pattern Recognition Letters. 27 (8): 861-874; Encyclopedia of Machine Learning and Data Mining, Claude Sammut, and Geoffrey I. Webb, eds. Springer (2017) and The Elements of Statistical Learning: Data Mining, Inference, and Prediction, Trevor Hastie, Robert Tibshirani, and Jerome Friedman, eds. 2^{nd} Edition Springer (2017).

This disclosure also utilizes routine methods in the field of bioinformatics. Basic texts disclosing the general methods and terms in bioinformatics include e.g., Current Protocols in Bioinformatics, Andreas D. Baxevanis and Daniel B. Davison eds. Wiley (2003).

### Sample Collection and Data Generation

Saliva sample can be collected from patients using any method known in the art e.g., using OMNIgene•ORAL (OM-505) kits. Metabolites isolated from saliva are quantitated and analyzed by any methods known in the art, for example using Gas Chromatography Mass Spectrometry (GCMS) or other mass spectrometry methods (see e.g., Mass Spectrometry, A Textbook (2020) Jürgen H. Gross, Springer (2006); Álvarez-Sánchez B., et al (2012). J. Chromatogr. A. 1248: 178-181; Sugimoto M., (2010) Metabolomics. 2010; 6:78-95).

### Differential Abundance of Metabolites and Salivary Metabolite Signature

Although well-codified, generally accepted methods for classifying the pathological progression of many forms of tumors are available today, the clinical classification of hepatocellular carcinoma and the correlated therapeutic indications entail very complex procedures and depend both on the degree of tumor progression as well as residual liver function, and thus it can be difficult to accurately determine if an individual has HCC and/or to distinguish whether a high risk patient such as an individual having cirrhosis has HCC. Therefore, of particular interest, and currently unmet need, is the ability to detect HCC in high-risk individuals, such as those with cirrhosis. Therefore, the identification of specific markers capable of accurately screening patients affected by hepatocellular carcinoma and distinguishing those patients from individuals having liver cirrhosis is an indispensable objective, especially because the ability to intervene when the disease is early can maximize the chance of patients being eligible for curative treatments.

The objective of a universally accepted staging is also potentially useful for improving the accuracy of the prognosis in individual patients, favoring the selection of patients for different therapies and, finally, adapting groups of patients based on therapeutic efficacy.

The identification of molecular biomarkers that function as a signature for detecting and differentiating HCC and cirrhosis offers hope of improving the diagnosis or prognosis of hepatocellular carcinoma, assessing the risk of developing hepatocellular carcinoma and monitoring the effectiveness of a therapeutic treatment against hepatocellular carcinoma.

In this context, the technical problem at the base of the present disclosure is to provide a method for detecting hepatocellular carcinoma and distinguishing HCC from liver cirrhosis and/or healthy individuals. The method is not invasive, is simple and fast, and at the same time accurate and reproducible, and is useful for assuring the choice of the best therapeutic treatment for each individual patient. For example, the method can be a factor in determining if a patient should be treated for HCC or not, or can be taken into consideration when deciding what follow-up tests should be done (e.g., biopsy), defining the response to therapies, monitoring any possible recurrences of the hepatocellular carcinoma, and identifying new therapeutic targets.

In the context of the present disclosure, the term “non-invasive” signifies the possibility, by means of a simple saliva test, of devising made-to-measure treatments for individual patients, as opposed to relying on disadvantageous methods with costly imaging and invasive biopsies, which at present represent the classic clinical approach for cancer diagnosis, prognosis and hence therapy. In particular, a specific panel of biomarkers, present and stable in the saliva, can be used as a molecular “fingerprint” of hepatocellular carcinoma and/or overall liver disease status.

The present disclosure relates to a method for diagnosing or prognosticating hepatocellular carcinoma, including early stage HCC, for assessing the risk of developing HCC or for monitoring the effectiveness of an anti-tumor therapy against HCC, which comprises measuring relative metabolite levels in saliva, for example by GCMS, and comparing said measured level of abundance in a subject with an appropriate reference level or control.

Thus, differential abundance of one or more salivary metabolites selected from the group comprising or consisting of acetophenone, octadecanol, lauric acid, 3-hydroxybutyric acid, 1-monopalmitin, 1-monostearin and combinations thereof is indicative of liver disease status. In some embodiments, differential abundance of one or more of salivary metabolites selected from acetophenone, octadecanol, lauric acid, 3-hydroxybutyric acid and combinations thereof compared to a reference level distinguishes a subject having HCC from a subject having liver cirrhosis.

Differential abundance of salivary metabolites compared to a reference level can be determined by any method known in the art, including, but not limited to use of mass spectrometry (see e.g., Xiaohang Wang and Liang Li,. (2020) Mass Spectrometry Letters Vol. 11, No. 2, 2020) and nuclear magnetic resonance (NMR) (see e.g., Dona, A. C. et al., (2016) Computational and Structural Biotechnology Journal Vol. 14: 135-153).

An alteration in the metabolite profile in a sample of a test subject, as compared to a control sample, may be indicative of the fact that the subject is affected by hepatocellular carcinoma or has an increased risk of developing hepatocellular carcinoma. Furthermore, an alteration in the level of abundance of metabolite in a sample of the test subject, as compared to a control sample, is indicative of the effectiveness, evolution and outcome of a therapy against hepatocellular carcinoma.

An alteration in the metabolite profile in a sample of the test subject, as compared to a control sample, may also be indicative of the evolution of the disease and hence of the prognosis thereof.

The methods disclosed herein can also be used to diagnose or assess the risk of developing HCC in liver cirrhosis patients affected, for example, by chronic hepatitis or in healthy subjects, or to prognosticate the evolution of cirrhosis in patients affected by cirrhosis, or to monitor the effectiveness of a pharmacological therapy against liver cirrhosis or to monitor the effectiveness of a pharmacological therapy to prevent or mitigate HCC.

In an exemplary embodiment, the method comprises measuring, for example by utilizing an appropriate mass spectrometry technique (see e.g., Jürgen H. Gross (2006) supra) in a saliva sample and comparing said measured level of abundance with a reference level. An alteration in the metabolite profile in a sample of the test subject, as compared to a control sample, is indicative of the fact that the subject is affected by HCC or has an increased risk of developing HCC, as for example in the case of patients affected by liver cirrhosis.

Such alteration may also indicative of the effectiveness, evolution and outcome of a therapy against liver cirrhosis to prevent further development to HCC.

The methods disclosed herein can be applied in combination with: microarrays, proteomic and immunological analyses, and sequencing analyses of specific DNA sequences for the purpose of defining an ad hoc therapeutic or diagnostic approach for individual patients. Completing the clinical information derived from known investigative techniques with that of the present disclosure would help to address the treatment of a patient affected by liver disease e.g., hepatocellular carcinoma or cirrhosis, in a completely personalized manner that is advantageous as regards both the diagnosis and the prognosis and therapy.

The metabolite profile as disclosed herein, harnesses the information of multiple biomarkers for identifying the pathology, defining the response to therapies and monitoring any disease status. Specific metabolite profiles as disclosed herein are also useful for defining the altered molecular pathways in hepatocellular carcinoma and can contribute, therefore, to identifying new therapeutic targets.

### Machine Learning and Artificial Intelligence

Machine learning methods can be used to model the likelihood of a salivary metabolite signature as predictive of the presence of HCC. Least absolute shrinkage and selection operator (LASSO) penalized logistic regression is an 11-penalized regression method that performs both regularization and variable selection, which results in a regression solution with improved interpretability and prediction accuracy compared to other regression approaches. LASSO is known in the art (see e.g., Tibshirani R. et al. J R Stat Soc Series B Stat Methodol. 1996; 1:267-88).

Random Forest includes an ensemble of decision trees and incorporates feature selection and interactions naturally in the learning process. Random Forest is known in the art (see e.g., Breiman, L. (2001): Random forests. Mach. Learn. 45, 5-32; Qi Y. (2012) Random Forest for Bioinformatics. In: Zhang C., Ma Y. (eds) Ensemble Machine Learning. Springer, Boston, MA. https://doi.org/10.1007/978-1-4419-9326-7_11).

Cross-validation may be used to evaluate predictive models by partitioning the original sample into a training set to train the model, and a test set to evaluate it. Cross-validation, is any of various model validation techniques for assessing how the results of a statistical analysis will generalize to an independent data set. Typically, a model is given a dataset of known data on which training is run (training dataset), and a dataset of unknown data against which the model is tested (called the validation dataset or testing set). The goal of cross-validation is to test the model's ability to predict new data that was not used in estimating it to give an insight on how the model will generalize to an independent dataset. Cross-validation is known in the art (see e.g., The Elements of Statistical Learning: Data Mining, Inference, and Prediction. By Trevor Hastie, Robert Tibshirani, and Jerome Friedman, Second Edition, Springer 2009.

Thus, in order to discover metabolic biomarkers that can discriminate, for example, between two or more clinical conditions, e.g. HCC and cirrhosis, the inventors applied a machine learning approach (e.g. Random Forest™, LASSO, ten-fold cross-validation, area under the receiver operating characteristic curve (AUC), sensitivity, specificity, positive predictive value (PPV), negative predictive value (NPV), and balanced accuracy) leading to an algorithm that is trained by reference data (i.e. data of reference salivary metabolite signatures from the two clinical conditions, e.g. HCC and cirrhosis, healthy and cirrhosis, etc., for the defined set of metabolic markers) to discriminate between statistical classes (i.e. two clinical conditions, e.g. HCC and cirrhosis).

As discussed in detail below, the inventors have identified metabolites in saliva that differed significantly in abundance among disease states and used machine-learning to discover combinations of metabolites with predictive power to accurately classify patients with HCC, patients with cirrhosis, and healthy individuals. Thus, the present inventors have discovered particular patterns of metabolite abundance providing high diagnostic accuracy, specificity and sensitivity in the determination of the HCC status of patients.

### Compounds and Therapies to Improve HCC

A subject diagnosed as having HCC or as being at elevated risk of HCC may be treated by any known method in the art, including being treated with palliative therapy.

Some liver problems can be treated with lifestyle modifications, such as stopping alcohol use, losing weight, or increasing exercise in combination with monitoring of liver function. Other liver problems may be treated with medications or may require surgery or a liver transplant.

The methods disclosed herein can be a factor in determining if a patient should be treated for HCC or not, or can be taken into consideration when deciding what follow-up tests should be done (e.g., biopsy), defining the response to therapies, monitoring any possible recurrences of the hepatocellular carcinoma, and identifying new therapeutic targets.

### Kits

In some embodiments, the disclosure provides kits comprising instructions for analyzing differentially abundant salivary metabolites as disclosed herein.

### Example

The following Example illustrates that a combination of four (4) metabolites detectable in a saliva sample can distinguish HCC, cirrhosis, and healthy patients. HCC was predicted with a sensitivity of 88% and specificity of 94%, resulting in balanced accuracy 91%. Cirrhosis was predicted with a sensitivity of 82% and specificity of 90% resulting in a balanced accuracy of 86%.

### Methods

### Patient Recruitment and Sample Collection

Saliva samples were collected from a real-world clinical cohort of 111 adult patients (>18 years of age) seen at the Cleveland Clinic (Cleveland, OH) between 2018-2020 with cirrhosis (N=30) or HCC (N=37) that underwent liver transplantation for HCC or cirrhosis, surgical resection for HCC, or liver biopsy with confirmed cirrhosis and/or HCC. In addition, patients attending treatment for hernia with no history of liver disease or liver cancer were used as healthy control subjects (N=43). Clinical characteristics of study participants can be found in Table 1. In addition to an initial assessment from imaging and clinical presentation, a histopathological assessment was performed to confirm HCC and cirrhosis diagnoses as part of the patient's standard of care. Written informed consent was provided by all participants, the study conformed to the ethical guidelines of the 1975 Declaration of Helsinki, and was approved by the Cleveland Clinic IRB (IRB #10-347).

TABLE 1
Summary statistics for study cohort

| Characteristic | Healthy | Cirrhosis | HCC |
|---|---|---|---|
| Total (n) | 43 | 30 | 37 |
| Mean age (min-max) | 57.6 (36-77) | 58 (33-80) | 67.3 (44-94) |
| Sex | | | |
| Male (%) | 27 (63%) | 12 (40%) | 30 (81%) |
| Female (%) | 16 (37%) | 18 (60%) | 7 (19%) |
| Diabetes mellitus type 2 | 11(26%) | 14(47%) | 21(57%) |
| Hypertension | 19(44%) | 13(43%) | 23(62%) |
| Coronary artery disease | 4(9%) | 6(20%) | 11(30%) |
| Hyperlipidemia | 14(33%) | 7(23%) | 12(32%) |
| Psychiatric disorder | 12(28%) | 7(23%) | 6(16%) |
| COPD/Asthma/OSA | 15(35%) | 6(20%) | 9(24%) |
| Other cancer history | 6(14%) | 6(20%) | 10(27%) |
| Thyroid | 7(16%) | 8(27%) | 2(5%) |
| Other PMH | 30(70%) | 29(97%) | 32(86%) |
| Ascites | 0(0%) | 21(70%) | 9(24%) |
| Encephalopathy | 0(0%) | 20(67%) | 7(19%) |
| Mean Hemoglobin (g/dl) (SEM) | 13.3 (0.3) | 11.2 (0.6) | 13.5 (0.4) |
| Mean Platelets (k/uL) (SEM) | 274.6 (12.2) | 116.4 (12.2) | 190.4 (13.8) |
| Mean AST (U/L) (SEM) | 24.1 (1.4) | 55.6 (7) | 73.5 (12.6) |
| Mean ALT (U/L) (SEM) | 24.3 (2.2) | 41.9 (9.7) | 58.2 (10.2) |
| Mean ALP (U/L) (SEM) | 79.1 (5.3) | 208.8 (35.5) | 128.4 (14) |
| Mean Bilirubin, Total (mg/dL) (SEM) | 0.5 (0.1) | 1.8 (0.3) | 1 (0.2) |
| Mean Albumin (g/dL) (SEM) | 4.2 (0.1) | 3.5 (0.1) | 3.8 (0.1) |
| Mean PT-INR (SEM) | 0.9 (0) | 1.3 (0.1) | 1.2 (0) |
| Mean Glucose (mg/dL) (SEM) | 114.2 (7.7) | 132.5 (14.8) | 138.3 (10.1) |
| Mean Creatinine (mg/dL) (SEM) | 1.1 (0.1) | 1 (0.1) | 1 (0.1) |

### Saliva Collection and Gas Chromatography Mass Spectrometry

A saliva sample was collected, after a standard mouth rinse, from each subject using the DNA Genotek OMNIgene ORAL OM-505 (Ottawa, Ontario) at the time of their scheduled visit with their physician. Samples were subjected to untargeted gas chromatography time of flight mass spectrometry (GC-TOF MS) at the West Coast Metabolomics Center (Davis, CA). A Leco Pegasus IV mass spectrometer was used with unit mass resolution at 17 spectra s-1 from 80-500 Da at −70 eV ionization energy and 1800 V detector voltage with a 230° C. transfer line and a 250° C. ion source. The analytical GC column was protected by a 10 m long empty guard column which is cut by 20 cm intervals whenever the reference mixture QC samples indicate problems caused by column contaminations. This chromatography method is designed to yield high quality retention and separation of primary metabolite classes (amino acids, hydroxyl acids, carbohydrates, sugar acids, sterols, aromatics, nucleosides, amines and other compounds) with narrow peak widths of 2-3 s and high quality within-series retention time reproducibility of better than 0.2 s absolute deviation of retention times. An automatic liner exchange was used after each set of 10 injections to reduce sample carryover for highly lipophilic compounds such as free fatty acids. Samples were run in two batches, resulting in 181 and 163 identified metabolites detected in each batch, respectively, and the relative abundance levels, quantified by peak height, were reported. 125 metabolites were identified in both batches and represented lipids, amino acids, peptides and sugars involved in pathways such as glycolysis, citric acid cycle, the urea cycle, fatty acid metabolism, phospholipid biosynthesis and ethanol degradation among others.

### Data Processing and Quality Control

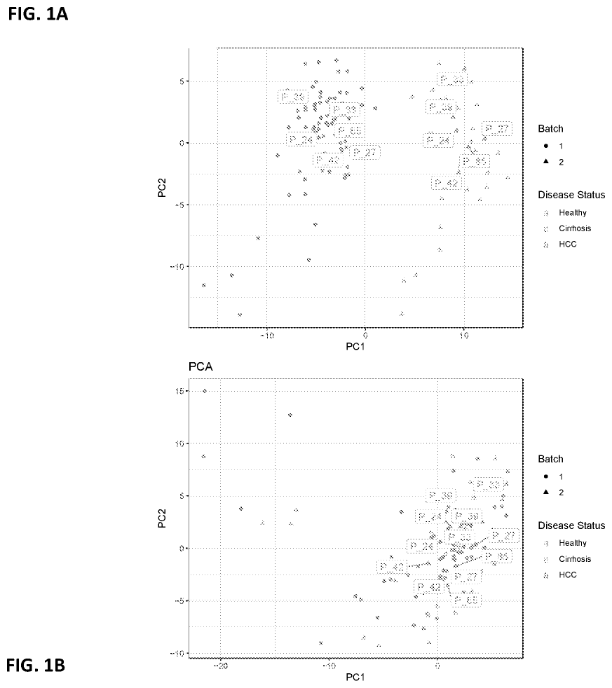

Missing values (two metabolites in three subjects) were imputed with half the minimum relative abundance across the cohort. Metabolite relative abundance levels were right skewed and log transformation was effective at normalizing the data. Six technical duplicate samples were included in each of the two experimental batches for quality control purposes. Principal component analysis (PCA) revealed variation in the metabolite relative abundance due to experimental batch as evidenced by the separation of these technical replicates. Mean centering and scaling by the metabolite standard deviations were effective for neutralizing differences due to batch (FIG. 1).

### Metabolite Associations

Metabolite associations with disease group were performed using the open source, statistical analysis software, R. The relative abundance levels of the 125 identified metabolites were individually tested for associations with disease status (i.e., healthy, cirrhosis, HCC) using pair-wise logistic regression models. Age, sex, and smoking status were tested for association using logistic regression models with each disease outcome and were included as model covariates when significantly associated with disease status (P<0.05) (Tables 2 and 3). All metabolite P values were adjusted for multiple testing using the Benjamini-Hochberg false discovery rate (FDR) approach and an FDR P<0.2 was used as the threshold for statistical significance.

TABLE 2
Disease status associations with age (days)

| Group1 (Reference) | Group2 | P Value | FDR P |
|---|---|---|---|
| Cirrhosis | Healthy | 0.862 | 0.862 |
| HCC | Healthy | 0.001 | 0.002 |
| HCC | Cirrhosis | 0.002 | 0.003 |

TABLE 3
Disease status associations with sex (male)

| Group1 (Reference) | Group2 | P Value | FDR P |
|---|---|---|---|
| Cirrhosis | Healthy | 0.057 | 0.076 |
| HCC | Healthy | 0.076 | 0.076 |
| HCC | Cirrhosis | 0.001 | 0.003 |

### Predictive Model Development

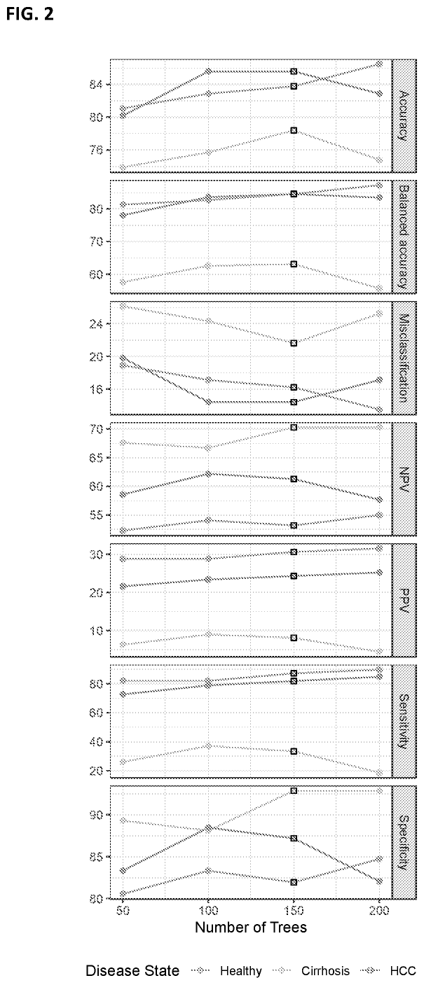

First, 10% of the subjects in each disease group were randomly partitioned into a test set that was excluded from model training and used to evaluate model performance. The remaining subjects (N=99) were assigned to the model training set. We evaluated four tree-based machine learning approaches to determine whether combinations of salivary metabolites could be used as a biomarker signature for detecting HCC and cirrhosis. To prevent model overfitting, each model was trained using a leave-one-out cross-validation (LOOCV) approach, where a single subject is iteratively removed from model training and then the model is used to make a prediction on the withheld subject. Model training performance was then evaluated on the withheld subjects from the LOOCV procedure and on the withheld test subjects. Three variations of Random Forest™ (RF)17 were investigated: 1) A random forest model (RF125) including all detected metabolites was used to classify subjects by disease status. Hyperparameter optimization was performed using a grid search to identify the optimal number of trees (ntree), and 150 was chosen as the optimal ntree value based on the mean misclassification, sensitivity, and specificity across the LOOCV iterations (FIG. 2). We then employed an iterative random forest approach (iRF) to select metabolites that would produce a model that would maximize predictive power with a minimal set of metabolites. This was done by generating a model using all 125 metabolites (RF125) and then iteratively eliminating the metabolite with the lowest mean Gini score across the LOOCV procedure until only a single metabolite remained. 2) The second model included twelve metabolites, representing the top 10% of metabolites selected using iRF approach (iRF12). 3) The out of bag error was then used to select the optimal number of metabolites that would produce the best performing model, and this model optimized at four metabolites (iRF4). 4) We also employed a classification and regression tree method (CART) to generate a binary decision tree to classify disease status based on metabolite abundance using the R package, rpart18. The 12 selected metabolites from iRF12 were used as input into the CART model, which was built using a LOOCV procedure for the 99 subjects in the training set. The CART model optimized at 4 metabolites (minsplit=20, cp=0.01). LOOCV was used to calculate sensitivity, specificity, balanced accuracy, misclassification, positive predictive value (PPV), and negative predictive value (NPV) for each of the four models. Each model was then evaluated for accuracy and overfitting using the withheld test cohort of 11 subjects (4 healthy, 3 cirrhosis, 4 HCC).

### Results

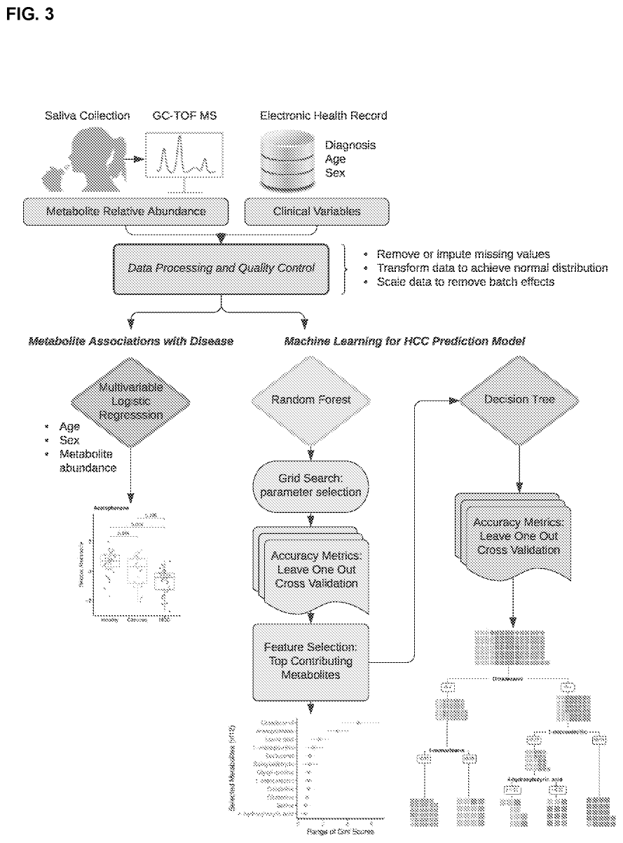

Out of the 110 participants (43 healthy, 30 cirrhosis, 37 HCC), a total of 125 metabolites were identified from obtained saliva samples (FIG. 3). There were some significant demographic differences between the groups, which were used as covariates to adjust for potential bias in the metabolite associations. Individuals in the HCC group were typically older than individuals in the cirrhosis and healthy groups (P<0.05). In addition, there were significantly more males in the HCC group than in the cirrhosis group (P<0.05). Lastly, current smoking status was significantly higher in patients with HCC than those with cirrhosis (P<0.05) (Tables 2-4).

TABLE 4
Disease status associations with Smoking Status

| Group1 (Reference) | Group2 | P Value | FDR P |
|---|---|---|---|
| Cirrhosis | Healthy | 0.992 | 0.992 |
| HCC | Healthy | 0.992 | 0.992 |
| HCC | Cirrhosis | 0.029 | 0.087 |

### Metabolite Associations

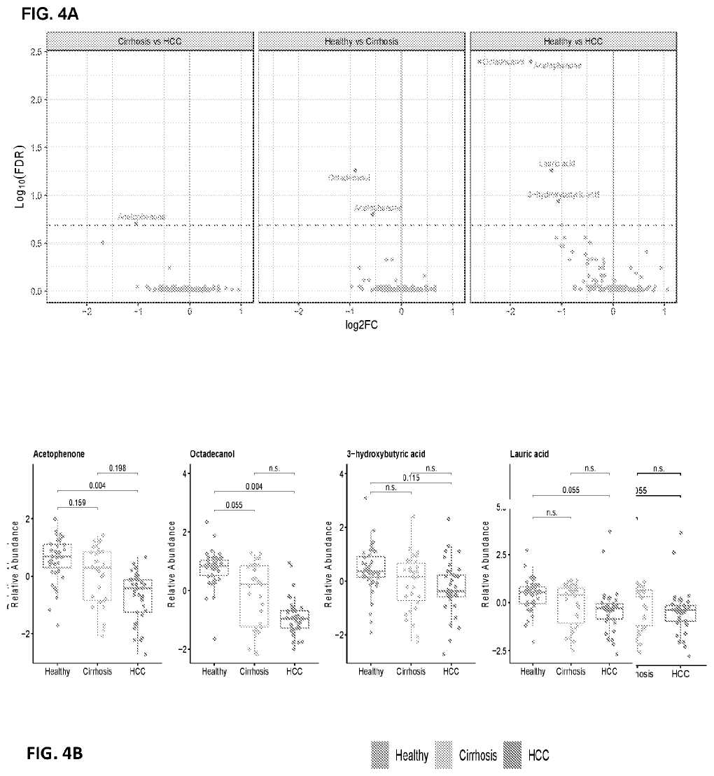

Four metabolites-acetophenone, octadecanol, lauric acid, 3-hydroxybutyric acid-were significantly different between two or more groups (FDR P<0.20) (FIGS. 4A and 4B Table 5). Acetophenone was significantly different in all three pair-wise comparisons: compared to healthy individuals, it was significantly decreased in patients with cirrhosis and significantly decreased further in patients with HCC. Octadecanol was also decreased in both and patients with HCC and patients with cirrhosis in comparison to healthy control subjects (FIGS. 4A and 4B Table 5). Additionally, lauric acid, 3-hydroxybutyric acid, threonic acid, glycerol-alpha-phosphate, butylamine and alpha-tocopherol were decreased in patients with HCC compared to healthy control subjects (FIGS. 4A and 4B, Table 5). Associations for all metabolites with each disease status are provided in Tables 6-8.

TABLE 5
Significant disease status associations with metabolite abundance

| Group1 (Reference) | Group2 | Metabolite | Coefficient (SE) | P Value | FDR P |
|---|---|---|---|---|---|
| Cirrhosis | HCC | Acetophenone | −1.124 (0.387) | 0.004 | 0.198 |
| Healthy | Cirrhosis | Acetophenone | −1.026 (0.34) | 0.003 | 0.159 |
| Healthy | Cirrhosis | Octadecanol | −1.498 (0.431) | <0.001 | 0.055 |
| Healthy | HCC | 3-hydroxybutyric acid | −1.299 (0.41) | 0.002 | 0.115 |
| Healthy | HCC | Acetophenone | −2.289 (0.531) | <0.001 | 0.004 |
| Healthy | HCC | Lauric acid | −1.062 (0.309) | <0.001 | 0.055 |
| Healthy | HCC | Octadecanol | −3.656 (0.861) | <0.001 | 0.004 |

TABLE 6
Associations of metabolites in Healthy (reference) versus Cirrhosis

| Metabolite | Coefficient SE | P Value | FDR P |
|---|---|---|---|
| 1-kestose | 0.03 (0.242) | 0.902 | 0.981 |
| 3-(4-hydroxyphenyl)propionic acid | −0.255 (0.305) | 0.404 | 0.946 |
| Proline | 0.157 (0.273) | 0.566 | 0.966 |
| Propane-1,3-diol | −0.335 (0.254) | 0.187 | 0.893 |
| Putrescine | 0.337 (0.284) | 0.234 | 0.893 |
| Pyruvic acid | 0.073 (0.297) | 0.805 | 0.981 |
| Salicylaldehyde | −0.158 (0.248) | 0.524 | 0.966 |
| Serine | −0.243 (0.278) | 0.383 | 0.943 |
| Sophorose | 0.008 (0.259) | 0.975 | 0.991 |
| Sorbitol | −0.077 (0.255) | 0.761 | 0.981 |
| Spermidine | −0.393 (0.268) | 0.144 | 0.893 |
| Squalene | −0.308 (0.243) | 0.205 | 0.893 |
| 3-aminoisobutyric acid | −0.021 (0.266) | 0.936 | 0.990 |
| Stearic acid | −0.112 (0.244) | 0.647 | 0.979 |
| Succinic acid | 0.191 (0.25) | 0.444 | 0.946 |
| Sucrose | −0.169 (0.257) | 0.509 | 0.966 |
| Threitol | −0.399 (0.291) | 0.171 | 0.893 |
| Threonic acid | −0.327 (0.264) | 0.215 | 0.893 |
| Threonine | 0.058 (0.25) | 0.816 | 0.981 |
| Thymine | 0.315 (0.275) | 0.252 | 0.893 |
| Tocopherol alpha- | −0.587 (0.294) | 0.046 | 0.572 |
| Tryptophan | −0.037 (0.259) | 0.886 | 0.981 |
| Tyrosine | 0.031 (0.242) | 0.900 | 0.981 |
| 3-hydroxybutyric acid | −0.63 (0.29) | 0.030 | 0.466 |
| Uracil | 0.031 (0.263) | 0.906 | 0.981 |
| Urea | −0.106 (0.255) | 0.678 | 0.980 |
| Uric acid | −0.185 (0.255) | 0.468 | 0.946 |
| Valine | 0.193 (0.27) | 0.474 | 0.946 |
| Xanthine | −0.297 (0.263) | 0.258 | 0.893 |
| Xylitol | −0.329 (0.257) | 0.201 | 0.893 |
| 3-phenyllactic acid | 0.21 (0.231) | 0.363 | 0.943 |
| 3-phosphoglycerate | 0.012 (0.266) | 0.964 | 0.991 |
| 4-aminobutyric acid | 0.101 (0.244) | 0.680 | 0.980 |
| 4-hydroxybutyric acid | −0.023 (0.23) | 0.922 | 0.985 |
| 4-hydroxyphenylacetic acid | 0.24 (0.256) | 0.348 | 0.943 |
| 4716 | −0.414 (0.275) | 0.132 | 0.893 |
| 5-aminovaleric acid | 0.19 (0.255) | 0.456 | 0.946 |
| 1-monopalmitin | −0.033 (0.243) | 0.891 | 0.981 |
| Acetophenone | −1.026 (0.34) | 0.003 | 0.159 |
| Adenine | −0.203 (0.253) | 0.421 | 0.946 |
| Adenosine-5-monophosphate | 0.119 (0.269) | 0.659 | 0.980 |
| Alanine | 0.084 (0.254) | 0.741 | 0.981 |
| Alanine-alanine | −0.239 (0.291) | 0.412 | 0.946 |
| Aminomalonate | −0.027 (0.264) | 0.920 | 0.985 |
| Arabitol | −0.183 (0.281) | 0.513 | 0.966 |
| Arachidic acid | −0.205 (0.274) | 0.456 | 0.946 |
| Asparagine | −0.209 (0.246) | 0.396 | 0.943 |
| Benzoic acid | −0.131 (0.248) | 0.596 | 0.966 |
| 1-monostearin | −0.23 (0.25) | 0.359 | 0.943 |
| Beta-glycerolphosphate | −0.295 (0.249) | 0.237 | 0.893 |
| Butane-2,3-diol | 0.476 (0.264) | 0.071 | 0.690 |
| Butylamine | −0.339 (0.259) | 0.191 | 0.893 |
| Butyrolactam | −0.098 (0.247) | 0.691 | 0.980 |
| Cellobiose | 0.029 (0.235) | 0.902 | 0.981 |
| Cerotinic acid | −0.067 (0.271) | 0.806 | 0.981 |
| Cholesterol | −0.412 (0.251) | 0.101 | 0.776 |
| Citramalic acid | 0.366 (0.264) | 0.166 | 0.893 |
| Citric acid | 0.03 (0.231) | 0.898 | 0.981 |
| Citrulline | −0.045 (0.231) | 0.846 | 0.981 |
| 1,5-anhydroglucitol | −0.174 (0.232) | 0.453 | 0.946 |
| Creatinine | −0.288 (0.253) | 0.254 | 0.893 |
| Cysteine | −0.274 (0.265) | 0.300 | 0.928 |
| Cystine | −0.126 (0.29) | 0.664 | 0.980 |
| D-erythro-sphingosine | −0.045 (0.238) | 0.849 | 0.981 |
| Dodecanol | −0.389 (0.268) | 0.147 | 0.893 |
| Fructose | −0.265 (0.27) | 0.325 | 0.943 |
| Fructose-6-phosphate | −0.236 (0.27) | 0.382 | 0.943 |
| Fucose | −0.007 (0.269) | 0.980 | 0.991 |
| Fumaric acid | 0.129 (0.246) | 0.600 | 0.966 |
| Galactinol | 0.128 (0.237) | 0.589 | 0.966 |
| 2-aminobutyric acid | 0.251 (0.257) | 0.329 | 0.943 |
| Glucose | −0.074 (0.259) | 0.775 | 0.981 |
| Glucose-1-phosphate | −0.139 (0.245) | 0.570 | 0.966 |
| Glucose-6-phosphate | −0.161 (0.257) | 0.531 | 0.966 |
| Glutamic acid | −0.017 (0.263) | 0.950 | 0.991 |
| Glutamine | −0.22 (0.268) | 0.410 | 0.946 |
| Glutaric acid | 0.255 (0.273) | 0.350 | 0.943 |
| Glyceric acid | 0.193 (0.245) | 0.431 | 0.946 |
| Glycerol | −0.532 (0.321) | 0.097 | 0.776 |
| Glycerol-alpha-phosphate | −0.436 (0.258) | 0.091 | 0.774 |
| Glycine | −0.066 (0.252) | 0.792 | 0.981 |
| 2-deoxypentitol | 0.034 (0.264) | 0.897 | 0.981 |
| Glycolic acid | −0.001 (0.224) | 0.998 | 0.998 |
| Glycyl tyrosine | 0.133 (0.25) | 0.596 | 0.966 |
| Glycyl-proline | −0.12 (0.283) | 0.673 | 0.980 |
| Guanidinosuccinate | −0.011 (0.266) | 0.968 | 0.991 |
| Guanine | −0.213 (0.249) | 0.392 | 0.943 |
| Heptadecanoic acid | −0.178 (0.243) | 0.463 | 0.946 |
| Homoserine | 0.278 (0.275) | 0.313 | 0.942 |
| Indole-3-acetate | 0.327 (0.277) | 0.237 | 0.893 |
| Inulotriose | −0.111 (0.283) | 0.696 | 0.980 |
| Isoleucine | 0.096 (0.267) | 0.720 | 0.981 |
| 2-hydroxyglutaric acid | 0.039 (0.28) | 0.890 | 0.981 |
| Isomaltose | 0.265 (0.251) | 0.291 | 0.928 |
| Isothreonic acid | −0.027 (0.255) | 0.915 | 0.983 |
| Lactic acid | −0.143 (0.244) | 0.557 | 0.966 |
| Lauric acid | −0.602 (0.279) | 0.031 | 0.466 |
| Leucine | −0.076 (0.253) | 0.765 | 0.981 |
| Levoglucosan | 0.015 (0.232) | 0.949 | 0.991 |
| Lysine | 0.108 (0.241) | 0.655 | 0.980 |
| Maleic acid | 0.136 (0.253) | 0.591 | 0.966 |
| Maleimide | −0.162 (0.269) | 0.546 | 0.966 |
| Malonic acid | −0.346 (0.267) | 0.195 | 0.893 |
| 2-ketoisocaproic acid | −0.229 (0.26) | 0.379 | 0.943 |
| Maltose | −0.309 (0.263) | 0.241 | 0.893 |
| Maltotriose | −0.143 (0.252) | 0.569 | 0.966 |
| Melezitose | 0.028 (0.251) | 0.910 | 0.981 |
| Methionine | 0.043 (0.232) | 0.853 | 0.981 |
| Methionine sulfoxide | −0.132 (0.248) | 0.595 | 0.966 |
| Myo-inositol | −0.148 (0.237) | 0.532 | 0.966 |
| Myristic acid | −0.251 (0.257) | 0.329 | 0.943 |
| N-carbamoylaspartate | 0.228 (0.266) | 0.392 | 0.943 |
| Nicotinic acid | 0.07 (0.257) | 0.786 | 0.981 |
| Octadecanol | −1.498 (0.431) | <0.001 | 0.055 |
| 2,5-dihydroxypyrazine | −0.121 (0.243) | 0.620 | 0.968 |
| Oleamide | −0.22 (0.242) | 0.364 | 0.943 |
| Oleic acid | −0.217 (0.245) | 0.376 | 0.943 |
| Ornithine | 0.095 (0.248) | 0.703 | 0.980 |
| Oxoproline | −0.114 (0.273) | 0.678 | 0.980 |
| Palmitic acid | −0.133 (0.244) | 0.587 | 0.966 |
| Phenylacetic acid | 0.19 (0.269) | 0.481 | 0.949 |
| Phenylalanine | −0.07 (0.244) | 0.775 | 0.981 |
| Phosphate | 0.269 (0.274) | 0.326 | 0.943 |
| Phosphoethanolamine | 0.204 (0.255) | 0.423 | 0.946 |
| Piperidone | 0.16 (0.27) | 0.553 | 0.966 |

TABLE 7
Associations of metabolites in Cirrhosis (reference) vs HCC

| Metabolite | Coefficient SE | P Value | FDR P |
|---|---|---|---|
| 1-kestose | 0.105 (0.266) | 0.694 | 0.980 |
| 3-(4-hydroxyphenyl)propionic acid | −0.105 (0.23) | 0.647 | 0.979 |
| Proline | 0.068 (0.278) | 0.806 | 0.981 |
| Propane-1,3-diol | −0.305 (0.326) | 0.349 | 0.943 |
| Putrescine | 0.336 (0.276) | 0.224 | 0.893 |
| Pyruvic acid | −0.002 (0.26) | 0.993 | 0.996 |
| Salicylaldehyde | −0.159 (0.283) | 0.574 | 0.966 |
| Serine | 0.038 (0.241) | 0.876 | 0.981 |
| Sophorose | −0.212 (0.261) | 0.416 | 0.946 |
| Sorbitol | −0.248 (0.288) | 0.389 | 0.943 |
| Spermidine | 0.065 (0.327) | 0.843 | 0.981 |
| Squalene | −0.353 (0.314) | 0.261 | 0.893 |
| 3-aminoisobutyric acid | 0.003 (0.275) | 0.991 | 0.996 |
| Stearic acid | −0.221 (0.281) | 0.433 | 0.946 |
| Succinic acid | 0.108 (0.283) | 0.702 | 0.980 |
| Sucrose | −0.103 (0.238) | 0.667 | 0.980 |
| Threitol | −0.165 (0.241) | 0.494 | 0.962 |
| Threonic acid | −0.343 (0.3) | 0.252 | 0.893 |
| Threonine | 0.302 (0.284) | 0.288 | 0.928 |
| Thymine | 0.358 (0.298) | 0.230 | 0.893 |
| Tocopherol alpha- | −0.361 (0.269) | 0.181 | 0.893 |
| Tryptophan | 0.144 (0.279) | 0.605 | 0.966 |
| Tyrosine | 0.277 (0.28) | 0.322 | 0.943 |
| 3-hydroxybutyric acid | −0.164 (0.317) | 0.605 | 0.966 |
| Uracil | 0.029 (0.256) | 0.910 | 0.981 |
| Urea | 0.1 (0.298) | 0.738 | 0.981 |
| Uric acid | −0.28 (0.272) | 0.302 | 0.928 |
| Valine | 0.208 (0.256) | 0.417 | 0.946 |
| Xanthine | 0.019 (0.271) | 0.943 | 0.991 |
| Xylitol | −0.28 (0.414) | 0.498 | 0.962 |
| 3-phenyllactic acid | 0.051 (0.287) | 0.859 | 0.981 |
| 3-phosphoglycerate | 0.157 (0.281) | 0.576 | 0.966 |
| 4-aminobutyric acid | 0.321 (0.295) | 0.277 | 0.909 |
| 4-hydroxybutyric acid | 0.02 (0.263) | 0.940 | 0.990 |
| 4-hydroxyphenylacetic acid | 0.207 (0.284) | 0.465 | 0.946 |
| 4716 | 0.066 (0.283) | 0.816 | 0.981 |
| 5-aminovaleric acid | 0.026 (0.277) | 0.926 | 0.985 |
| 1-monopalmitin | −0.085 (0.266) | 0.751 | 0.981 |
| Acetophenone | −1.124 (0.387) | 0.004 | 0.198 |
| Adenine | 0.02 (0.261) | 0.939 | 0.990 |
| Adenosine-5-monophosphate | 0.167 (0.27) | 0.535 | 0.966 |
| Alanine | 0.356 (0.3) | 0.235 | 0.893 |
| Alanine-alanine | −0.21 (0.284) | 0.460 | 0.946 |
| Aminomalonate | 0.293 (0.278) | 0.292 | 0.928 |
| Arabitol | 0.003 (0.23) | 0.989 | 0.996 |
| Arachidic acid | 0.168 (0.289) | 0.562 | 0.966 |
| Asparagine | −0.052 (0.264) | 0.842 | 0.981 |
| Benzoic acid | 0.073 (0.281) | 0.796 | 0.981 |
| 1-monostearin | −0.226 (0.266) | 0.397 | 0.943 |
| Beta-glycerolphosphate | −0.014 (0.275) | 0.959 | 0.991 |
| Butane-2,3-diol | 0.074 (0.266) | 0.779 | 0.981 |
| Butylamine | −0.356 (0.3) | 0.235 | 0.893 |
| Butyrolactam | −0.272 (0.304) | 0.371 | 0.943 |
| Cellobiose | −0.051 (0.336) | 0.879 | 0.981 |
| Cerotinic acid | −0.425 (0.318) | 0.181 | 0.893 |
| Cholesterol | 0.008 (0.258) | 0.975 | 0.991 |
| Citramalic acid | 0.048 (0.27) | 0.860 | 0.981 |
| Citric acid | −0.082 (0.277) | 0.768 | 0.981 |
| Citrulline | 0.11 (0.293) | 0.708 | 0.981 |
| 1,5-anhydroglucitol | 0.238 (0.306) | 0.437 | 0.946 |
| Creatinine | 0.07 (0.256) | 0.784 | 0.981 |
| Cysteine | 0.189 (0.263) | 0.474 | 0.946 |
| Cystine | 0.078 (0.269) | 0.772 | 0.981 |
| D-erythro-sphingosine | −0.051 (0.287) | 0.859 | 0.981 |
| Dodecanol | −0.169 (0.269) | 0.531 | 0.966 |
| Fructose | −0.117 (0.251) | 0.640 | 0.979 |
| Fructose-6-phosphate | −0.192 (0.266) | 0.471 | 0.946 |
| Fucose | −0.044 (0.268) | 0.871 | 0.981 |
| Fumaric acid | 0.154 (0.266) | 0.561 | 0.966 |
| Galactinol | −0.735 (0.37) | 0.047 | 0.572 |
| 2-aminobutyric acid | −0.038 (0.25) | 0.881 | 0.981 |
| Glucose | −0.144 (0.25) | 0.566 | 0.966 |
| Glucose-1-phosphate | 0.082 (0.316) | 0.796 | 0.981 |
| Glucose-6-phosphate | −0.061 (0.253) | 0.808 | 0.981 |
| Glutamic acid | 0.124 (0.262) | 0.636 | 0.979 |
| Glutamine | −0.225 (0.274) | 0.412 | 0.946 |
| Glutaric acid | 0.132 (0.262) | 0.615 | 0.968 |
| Glyceric acid | 0.266 (0.295) | 0.368 | 0.943 |
| Glycerol | −0.191 (0.254) | 0.451 | 0.946 |
| Glycerol-alpha-phosphate | −0.158 (0.291) | 0.586 | 0.966 |
| Glycine | 0.201 (0.289) | 0.488 | 0.958 |
| 2-deoxypentitol | 0.086 (0.267) | 0.748 | 0.981 |
| Glycolic acid | −0.035 (0.308) | 0.909 | 0.981 |
| Glycyl tyrosine | −0.409 (0.313) | 0.191 | 0.893 |
| Glycyl-proline | −0.366 (0.291) | 0.208 | 0.893 |
| Guanidinosuccinate | 0.066 (0.249) | 0.792 | 0.981 |
| Guanine | −0.193 (0.296) | 0.513 | 0.966 |
| Heptadecanoic acid | −0.064 (0.264) | 0.809 | 0.981 |
| Homoserine | 0.235 (0.252) | 0.351 | 0.943 |
| Indole-3-acetate | 0.071 (0.269) | 0.791 | 0.981 |
| Inulotriose | −0.008 (0.252) | 0.976 | 0.991 |
| Isoleucine | 0.085 (0.253) | 0.736 | 0.981 |
| 2-hydroxyglutaric acid | 0.066 (0.263) | 0.803 | 0.981 |
| Isomaltose | −0.172 (0.278) | 0.537 | 0.966 |
| Isothreonic acid | 0.213 (0.23) | 0.355 | 0.943 |
| Lactic acid | 0.2 (0.275) | 0.467 | 0.946 |
| Lauric acid | −0.344 (0.275) | 0.211 | 0.893 |
| Leucine | 0.178 (0.282) | 0.528 | 0.966 |
| Levoglucosan | 0.1 (0.274) | 0.715 | 0.981 |
| Lysine | 0.174 (0.293) | 0.552 | 0.966 |
| Maleic acid | 0.125 (0.265) | 0.636 | 0.979 |
| Maleimide | −0.04 (0.244) | 0.869 | 0.981 |
| Malonic acid | −0.223 (0.292) | 0.446 | 0.946 |
| 2-ketoisocaproic acid | 0.323 (0.257) | 0.209 | 0.893 |
| Maltose | 0.007 (0.255) | 0.977 | 0.991 |
| Maltotriose | −0.066 (0.262) | 0.802 | 0.981 |
| Melezitose | −0.068 (0.282) | 0.808 | 0.981 |
| Methionine | 0.058 (0.278) | 0.836 | 0.981 |
| Methionine sulfoxide | 0.116 (0.301) | 0.701 | 0.980 |
| Myo-inositol | −0.13 (0.261) | 0.618 | 0.968 |
| Myristic acid | −0.316 (0.276) | 0.252 | 0.893 |
| N-carbamoylaspartate | 0.328 (0.348) | 0.346 | 0.943 |
| Nicotinic acid | 0.011 (0.253) | 0.967 | 0.991 |
| Octadecanol | −1.012 (0.397) | 0.011 | 0.312 |
| 2,5-dihydroxypyrazine | −0.125 (0.27) | 0.643 | 0.979 |
| Oleamide | 0.067 (0.303) | 0.825 | 0.981 |
| Oleic acid | −0.315 (0.29) | 0.278 | 0.909 |
| Ornithine | 0.067 (0.271) | 0.804 | 0.981 |
| Oxoproline | 0.095 (0.233) | 0.682 | 0.980 |
| Palmitic acid | −0.265 (0.284) | 0.351 | 0.943 |
| Phenylacetic acid | 0.139 (0.264) | 0.598 | 0.966 |
| Phenylalanine | 0.062 (0.273) | 0.821 | 0.981 |
| Phosphate | −0.239 (0.276) | 0.387 | 0.943 |
| Phosphoethanolamine | 0.006 (0.261) | 0.980 | 0.991 |
| Piperidone | 0.227 (0.289) | 0.432 | 0.946 |

TABLE 8
Associations of metabolites in Healthy (reference) vs HCC

| Metabolite | Coefficient SE | P Value | FDR P |
|---|---|---|---|
| 1-kestose | −0.077 (0.241) | 0.748 | 0.981 |
| 3-(4-hydroxyphenyl)propionic acid | −0.342 (0.267) | 0.201 | 0.893 |
| Proline | 0.063 (0.245) | 0.799 | 0.981 |
| Propane-1,3-diol | −0.582 (0.281) | 0.038 | 0.513 |
| Putrescine | 0.742 (0.315) | 0.019 | 0.388 |
| Pyruvic acid | −0.035 (0.243) | 0.887 | 0.981 |
| Salicylaldehyde | −0.265 (0.237) | 0.263 | 0.893 |
| Serine | −0.306 (0.256) | 0.232 | 0.893 |
| Sophorose | −0.38 (0.254) | 0.135 | 0.893 |
| Sorbitol | −0.272 (0.291) | 0.350 | 0.943 |
| Spermidine | −0.78 (0.329) | 0.018 | 0.388 |
| Squalene | −0.643 (0.263) | 0.014 | 0.338 |
| 3-aminoisobutyric acid | −0.102 (0.252) | 0.686 | 0.980 |
| Stearic acid | −0.299 (0.251) | 0.234 | 0.893 |
| Succinic acid | 0.14 (0.244) | 0.566 | 0.966 |
| Sucrose | −0.284 (0.233) | 0.223 | 0.893 |
| Threitol | −0.66 (0.296) | 0.026 | 0.466 |
| Threonic acid | −1.081 (0.379) | 0.004 | 0.202 |
| Threonine | 0.274 (0.266) | 0.302 | 0.928 |
| Thymine | 0.571 (0.292) | 0.051 | 0.572 |
| Tocopherol alpha- | −0.732 (0.276) | 0.008 | 0.276 |
| Tryptophan | 0.011 (0.254) | 0.964 | 0.991 |
| Tyrosine | 0.274 (0.245) | 0.262 | 0.893 |
| 3-hydroxybutyric acid | −1.299 (0.41) | 0.002 | 0.115 |
| Uracil | 0.1 (0.245) | 0.684 | 0.980 |
| Urea | −0.195 (0.248) | 0.431 | 0.946 |
| Uric acid | −0.478 (0.246) | 0.052 | 0.572 |
| Valine | 0.377 (0.256) | 0.140 | 0.893 |
| Xanthine | −0.362 (0.272) | 0.184 | 0.893 |
| Xylitol | −0.65 (0.381) | 0.088 | 0.767 |
| 3-phenyllactic acid | 0.289 (0.245) | 0.238 | 0.893 |
| 3-phosphoglycerate | 0.034 (0.244) | 0.888 | 0.981 |
| 4-aminobutyric acid | 0.108 (0.256) | 0.674 | 0.980 |
| 4-hydroxybutyric acid | −0.126 (0.243) | 0.603 | 0.966 |
| 4-hydroxyphenylacetic acid | 0.476 (0.269) | 0.076 | 0.715 |
| 4716 | −0.548 (0.277) | 0.048 | 0.572 |
| 5-aminovaleric acid | 0.107 (0.249) | 0.667 | 0.980 |
| 1-monopalmitin | −0.093 (0.237) | 0.693 | 0.980 |
| Acetophenone | −2.289 (0.531) | <0.001 | 0.004 |
| Adenine | −0.315 (0.251) | 0.210 | 0.893 |
| Adenosine-5-monophosphate | 0.255 (0.26) | 0.327 | 0.943 |
| Alanine | 0.242 (0.266) | 0.362 | 0.943 |
| Alanine-alanine | −0.766 (0.312) | 0.014 | 0.338 |
| Aminomalonate | 0.147 (0.243) | 0.546 | 0.966 |
| Arabitol | −0.371 (0.257) | 0.149 | 0.893 |
| Arachidic acid | −0.095 (0.263) | 0.716 | 0.981 |
| Asparagine | −0.43 (0.266) | 0.106 | 0.794 |
| Benzoic acid | −0.275 (0.254) | 0.279 | 0.909 |
| 1-monostearin | −0.431 (0.261) | 0.098 | 0.776 |
| Beta-glycerolphosphate | −0.293 (0.243) | 0.227 | 0.893 |
| Butane-2,3-diol | 0.406 (0.264) | 0.124 | 0.891 |
| Butylamine | −0.761 (0.287) | 0.008 | 0.276 |
| Butyrolactam | −0.363 (0.256) | 0.156 | 0.893 |
| Cellobiose | −0.325 (0.291) | 0.264 | 0.893 |
| Cerotinic acid | −0.52 (0.278) | 0.061 | 0.654 |
| Cholesterol | −0.426 (0.247) | 0.085 | 0.767 |
| Citramalic acid | 0.283 (0.252) | 0.262 | 0.893 |
| Citric acid | −0.045 (0.239) | 0.849 | 0.981 |
| Citrulline | −0.077 (0.241) | 0.750 | 0.981 |
| 1,5-anhydroglucitol | −0.088 (0.248) | 0.722 | 0.981 |
| Creatinine | −0.298 (0.246) | 0.225 | 0.893 |
| Cysteine | −0.171 (0.252) | 0.496 | 0.962 |
| Cystine | −0.205 (0.261) | 0.432 | 0.946 |
| D-erythro-sphingosine | −0.307 (0.257) | 0.232 | 0.893 |
| Dodecanol | −0.525 (0.285) | 0.066 | 0.684 |
| Fructose | −0.621 (0.289) | 0.032 | 0.466 |
| Fructose-6-phosphate | −0.512 (0.307) | 0.096 | 0.776 |
| Fucose | −0.167 (0.237) | 0.480 | 0.949 |
| Fumaric acid | 0.177 (0.232) | 0.446 | 0.946 |
| Galactinol | −0.526 (0.321) | 0.101 | 0.776 |
| 2-aminobutyric acid | 0.287 (0.262) | 0.272 | 0.909 |
| Glucose | −0.596 (0.273) | 0.029 | 0.466 |
| Glucose-1-phosphate | −0.126 (0.254) | 0.619 | 0.968 |
| Glucose-6-phosphate | −0.301 (0.247) | 0.222 | 0.893 |
| Glutamic acid | 0.037 (0.239) | 0.876 | 0.981 |
| Glutamine | −0.655 (0.306) | 0.032 | 0.466 |
| Glutaric acid | 0.565 (0.287) | 0.049 | 0.572 |
| Glyceric acid | 0.176 (0.271) | 0.515 | 0.966 |
| Glycerol | −1.001 (0.455) | 0.028 | 0.466 |
| Glycerol-alpha-phosphate | −0.727 (0.272) | 0.007 | 0.276 |
| Glycine | 0.071 (0.25) | 0.777 | 0.981 |
| 2-deoxypentitol | 0.044 (0.253) | 0.861 | 0.981 |
| Glycolic acid | −0.16 (0.253) | 0.527 | 0.966 |
| Glycyl tyrosine | −0.487 (0.27) | 0.072 | 0.690 |
| Glycyl-proline | −0.824 (0.326) | 0.012 | 0.312 |
| Guanidinosuccinate | −0.101 (0.236) | 0.667 | 0.980 |
| Guanine | −0.631 (0.303) | 0.037 | 0.513 |
| Heptadecanoic acid | −0.308 (0.242) | 0.203 | 0.893 |
| Homoserine | 0.3 (0.243) | 0.218 | 0.893 |
| Indole-3-acetate | 0.314 (0.262) | 0.231 | 0.893 |
| Inulotriose | −0.249 (0.247) | 0.314 | 0.942 |
| Isoleucine | 0.258 (0.248) | 0.297 | 0.928 |
| 2-hydroxyglutaric acid | −0.035 (0.242) | 0.884 | 0.981 |
| Isomaltose | 0.166 (0.251) | 0.508 | 0.966 |
| Isothreonic acid | −0.033 (0.231) | 0.887 | 0.981 |
| Lactic acid | −0.154 (0.239) | 0.519 | 0.966 |
| Lauric acid | −1.062 (0.309) | <0.001 | 0.055 |
| Leucine | 0.124 (0.249) | 0.618 | 0.968 |
| Levoglucosan | 0.148 (0.248) | 0.550 | 0.966 |
| Lysine | 0.181 (0.248) | 0.466 | 0.946 |
| Maleic acid | 0.059 (0.234) | 0.801 | 0.981 |
| Maleimide | −0.396 (0.25) | 0.113 | 0.834 |
| Malonic acid | −0.488 (0.267) | 0.067 | 0.684 |
| 2-ketoisocaproic acid | −0.07 (0.247) | 0.778 | 0.981 |
| Maltose | −0.314 (0.243) | 0.196 | 0.893 |
| Maltotriose | −0.212 (0.243) | 0.383 | 0.943 |
| Melezitose | −0.028 (0.243) | 0.910 | 0.981 |
| Methionine | 0.022 (0.243) | 0.927 | 0.985 |
| Methionine sulfoxide | −0.225 (0.254) | 0.377 | 0.943 |
| Myo-inositol | −0.328 (0.249) | 0.188 | 0.893 |
| Myristic acid | −0.71 (0.281) | 0.011 | 0.312 |
| N-carbamoylaspartate | 0.061 (0.252) | 0.810 | 0.981 |
| Nicotinic acid | −0.209 (0.246) | 0.394 | 0.943 |
| Octadecanol | −3.656 (0.861) | <0.001 | 0.004 |
| 2,5-dihydroxypyrazine | −0.434 (0.254) | 0.088 | 0.767 |
| Oleamide | −0.256 (0.249) | 0.305 | 0.928 |
| Oleic acid | −0.622 (0.269) | 0.021 | 0.412 |
| Ornithine | 0.111 (0.243) | 0.647 | 0.979 |
| Oxoproline | −0.106 (0.228) | 0.644 | 0.979 |
| Palmitic acid | −0.362 (0.257) | 0.159 | 0.893 |
| Phenylacetic acid | 0.287 (0.254) | 0.258 | 0.893 |
| Phenylalanine | 0.008 (0.243) | 0.975 | 0.991 |
| Phosphate | 0.065 (0.286) | 0.819 | 0.981 |
| Phosphoethanolamine | 0.315 (0.251) | 0.209 | 0.893 |
| Piperidone | 0.393 (0.266) | 0.139 | 0.893 |

Metabolite Selection Using Iterative Random Forest (iRF) and Decision Tree (DT) Approaches

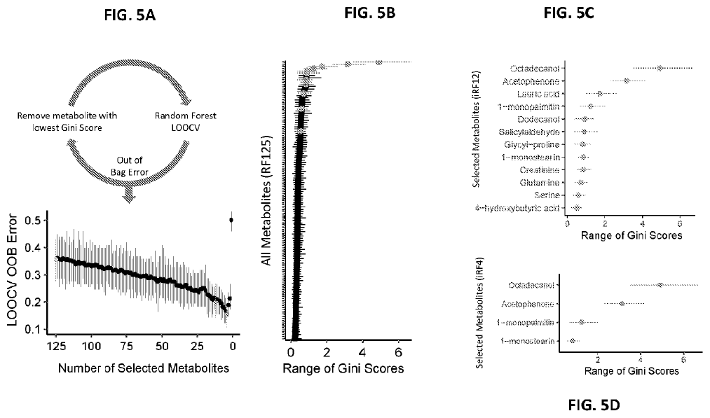

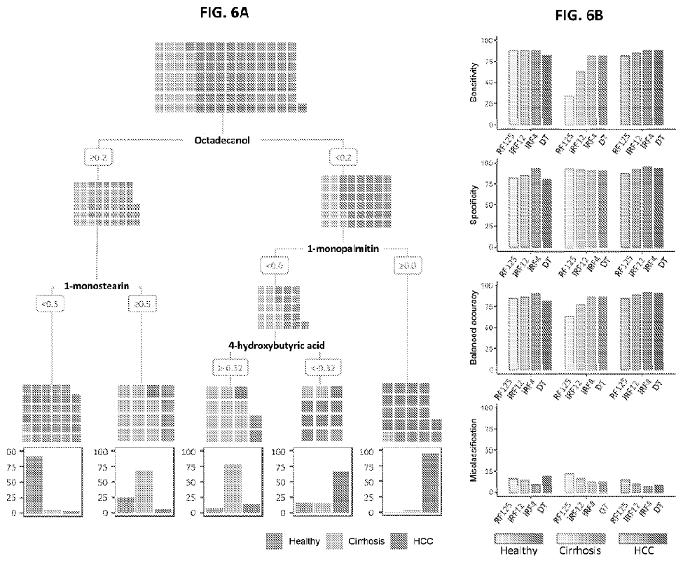

Three RF models were considered based on their mean training LOOCV out of bag (OOB) error rates. The initial model, incorporating all 125 metabolites (RF125) had a mean LOOCV OOB error rate of 35.6% and the range of Gini Scores, demonstrating metabolite importance, across LOOCV iterations for the 125 metabolites is shown in FIGS. 5A and 5B. A subsequent model, iRF12, included the top 10% of metabolites (n=12) selected using the iterative RF approach (FIG. 3A), and had a mean LOOCV OOB error of 19.7% (FIG. 3A,C). iRF4 was the model with the lowest global mean misclassification (15.3%) which utilized the following four metabolites—octadecanol, acetophenone, 1-monopalmitin and 1-monostearin (FIGS. 5A and 5D). A decision tree classification model was developed with the 12 metabolites selected for iRF12 (FIG. 5D). The pruned decision tree selected four metabolites octadecanol, 1-monopalmatin, 1-monostearin, and 4-hydroxybutyric acid—and had a LOOCV OOB error rate of 12.7% of the subjects (FIG. 6).

### Comparison of Model Performance

RF125 correctly classified 65/99 (66%) patients in the training cohort and 10/11 (91%) patients in the test cohort. iRF12 correctly classified 82/99 (83%) patients in the training cohort and 10/11 (91%) of patients in the test cohort. iRF4 correctly classified 85/99 (86%) patients in the training cohort and 9/11 (82%) of patients in the test cohort. The decision tree model correctly classified 83/99 (84%) patients in the training cohort and 8/11 (73%) patients in the test cohort (FIG. 6A). All models produced similar accuracy metrics in the training and test cohorts indicating minimal model overfitting. We also compared the performance metrics (i.e., sensitivity, specificity, balanced accuracy, misclassification, NPV, PPV) derived from the LOOCV of the training set across the four models for each disease status. Upon taking the mean of each metric among healthy, cirrhosis, and HCC, iRF4 outperformed other models in all metrics (Table 9).

TABLE 9
Accuracy metrics for predicting disease status

| | | | | Balanced | | | |
|---|---|---|---|---|---|---|---|
| Disease | Model | Sensitivity | Specificity | accuracy | Misclassification | PPV | NPV |
| Healthy | iRF125 | 87.2 | 81.9 | 84.6 | 16.2 | 30.6 | 53.1 |
| HCC | iRF125 | 81.8 | 87.2 | 84.5 | 14.4 | 24.3 | 61.3 |
| Cirrhosis | iRF125 | 33.3 | 92.9 | 63.1 | 21.6 | 8.1 | 70.3 |
| Average | iRF125 | 67.4 | 87.3 | 77.4 | 17.4 | 21 | 61.6 |
| Healthy | iRF12 | 87.2 | 85 | 86.1 | 14.1 | 34.3 | 51.5 |
| HCC | iRF12 | 84.8 | 92.4 | 88.6 | 10.1 | 28.3 | 61.6 |
| Cirrhosis | iRF12 | 63 | 91.7 | 77.3 | 16.2 | 17.2 | 66.7 |
| Average | iRF12 | 78.3 | 89.7 | 84 | 13.5 | 26.6 | 59.9 |
| Healthy | iRF4 | 87.2 | 93.3 | 90.3 | 9.1 | 34.3 | 56.6 |
| HCC | iRF4 | 87.9 | 95.4 | 91.7 | 7.1 | 29.3 | 63.6 |
| Cirrhosis | iRF4 | 81.5 | 90.3 | 85.9 | 12.1 | 22.2 | 65.7 |
| Average | iRF4 | 85.5 | 93 | 89.3 | 9.4 | 28.6 | 62 |
| Healthy | Decision Tree | 82 | 80.3 | 81.2 | 19.1 | 29.1 | 51.8 |
| HCC | Decision Tree | 87.9 | 93.5 | 90.7 | 8.2 | 26.4 | 65.4 |
| Cirrhosis | Decision Tree | 81.5 | 90.4 | 85.9 | 11.8 | 20 | 68.2 |
| Average | Decision Tree | 83.8 | 88.1 | 85.9 | 13 | 25.2 | 61.8 |

### Healthy Subjects

For healthy subjects, specificity (93.3%), balanced accuracy (90.3%), PPV (34.3%), and NPV (56.6%) were highest, and misclassification (9.1%) was lowest, in model iRF4 (FIG. 6B, Table 9). Sensitivity was 87.2% across models RF125, iRF12 and iRF4.

### Cirrhosis

For patients with cirrhosis, balanced accuracy (85.9%) was highest and misclassification was lowest (11.8%) in the DT model. PPV (22.2%) was highest in model iRF4 and NPV was highest (70.3% in model iRF125). Sensitivity was highest in both the iRF4 and Decision Tree models (81.5%) (FIG. 6B, Table 9).

### HCC

For patients with HCC, specificity (95.4%), balanced accuracy (91.7%), and PPV (29.3%) were highest, and misclassification (7.1%) was lowest, in the iRF4 model. NPV (65.5%) was highest in DT model and sensitivity (87.9%) was highest in both iRF4 and DT models (FIG. 6B, Table 9).

### CONCLUSION

We interrogated four different tree-based machine-learning models to identify the panel of metabolites with the best predictive power. The four models, RF125, iRF12, iRF4 and DT displayed cross-validated sensitivities for detecting HCC of 81.8%, 84.9%, 87.9%, 87.9% and specificities of 87.2%, 92.4%, 95.5%, 93.5%, respectively. All models displayed better sensitivities and specificities across LOOCV than those reported by a meta-analysis of AFP (20-100 ng/ml) (61%, 86%) and AFP plus ultrasound (62%, 88%).

While certain embodiments of the present invention have been shown and described herein, it will be obvious to ordinarily skilled artisans that these embodiments are merely exemplary. Numerous variations, changes, and substitutions will occur to ordinarily skilled artisans within the scope and spirit of the invention. Various alternatives to the described embodiments may be employed. Accordingly, the invention should be considered as limited only by the scope of the following claims, and that methods and structures within the scope of these claims and their equivalents are covered.

What is claimed is:

1. A method for diagnosing or prognosticating hepatocellular carcinoma (HCC) in a subject, or for assessing the risk of developing HCC, or for monitoring the effectiveness of a therapy for HCC, comprising:
determining, in an isolated sample of saliva, the abundance of at least one salivary metabolite selected from the group consisting of acetophenone, octadecanol, lauric acid, 3-hydroxybutyric acid, 1-monopalmitin, 1-monostearin and combinations thereof, and determining whether the at least one salivary metabolite is differentially abundant compared to a reference sample, wherein differential abundance of the at least one salivary metabolite is an increase or a decrease, in order to determine the HCC status of the subject, and
treating the subject diagnosed with HCC or having elevated risk of HCC or as needing more effective treatment for HCC, with a compound or other therapy to improve the HCC.

2. The method of claim 1, wherein the reference sample is from a subject who does not have HCC.

3. The method of claim 2, wherein the reference sample is from a subject who has cirrhosis, or other form of chronic liver disease, such as fibrosis, steatosis, alcoholic steatohepatitis, or non-alcoholic steatohepatitis (NASH).

4. The method of claim 3, wherein the abundance of acetophenone in the subject is decreased as compared to the reference sample, and the subject is diagnosed as having HCC.

5. The method of claim 1, wherein the reference sample is from a healthy subject.

6. The method of claim 5, wherein the abundance of acetophenone in the subject is decreased as compared to the reference sample and the subject is diagnosed as having cirrhosis or HCC.

7. The method of claim 6, wherein the reference sample is from a subject who has cirrhosis, the abundance of acetophenone in the subject is decreased as compared to the reference sample, and the subject is diagnosed as having HCC.

8. The method of claim 5, wherein the abundance of octadecanol is decreased compared to the reference sample and the subject is diagnosed as having cirrhosis or HCC.

9. The method of claim 8, wherein the reference sample is from a subject who has cirrhosis and the subject is diagnosed as having HCC.

10. The method of claim 5, wherein the method has sensitivity of at least 81% and specificity of at least 87%.

11. A method for differentiating a subject having hepatocellular carcinoma (HCC) from a subject having liver cirrhosis, the method comprising the step of determining, in an isolated sample of saliva, the abundance of at least one salivary metabolite selected from the group consisting of acetophenone, octadecanol, lauric acid, 3-hydroxybutyric acid, 1-monopalmitin, 1-monostearin and combinations thereof, and
determining whether the at least one salivary metabolite is differentially abundant compared to a reference sample, wherein differential abundance of the at least one salivary metabolite is an increase or a decrease, and wherein differential abundance of the at least one salivary metabolite indicates the subject has HCC.

12. The method of claim 11, wherein the at least one salivary metabolite is acetophenone.

13. The method of claim 12, wherein the at least one salivary metabolite further includes octadecanol, 1-monopalmitin, and 1-monostearin.

14. The method of claim 11, wherein the at least one salivary metabolite is at least four salivary metabolites.

15. The method of claim 14, wherein when the four salivary metabolites include acetophenone, or octadecanol the four salivary metabolites do not include lauric acid, or 3-hydroxybutyric acid.

16. The method of claim 15, wherein the at least four metabolites are acetophenone, octadecanol, 1-monopalmitin, 1-monostearin.

17. The method of claim 15, wherein the at least four salivary metabolites are lauric acid, 3-hydroxybutyric acid, 1-monopalmitin, and 1-monostearin.

18. The method of any one of claims 11 to 17, comprising the additional step of treating the subject diagnosed with HCC with a compound or other therapy.

19. A method for discovering salivary biomarkers of hepatocellular carcinoma (HCC) in saliva from a subject having HCC, the method comprising:
(a) obtaining or having obtained a saliva sample from the subject, and a saliva sample from a subject not having HCC, and
(b) detecting metabolites that are differentially abundant in the subject having HCC compared to the subject not having HCC, wherein the differentially abundant metabolites have a high predictive value for detection of HCC, and wherein the detection step comprises machine learning that utilizes Random Forest™ and least absolute shrinkage and selection operator (LASSO) and cross-validation, wherein differentially abundant salivary metabolites are biomarkers of HCC.

20. The method of claim 19, wherein the differentially abundant salivary metabolites are selected from the group consisting of: 1-kestose, 3-(4-hydroxyphenyl)propionic acid, Proline, Propane-1,3-diol, Putrescine, Pyruvic acid, Salicylaldehyde, Serine, Sophorose, Sorbitol, Spermidine, Squalene, 3-aminoisobutyric acid, Stearic acid, Succinic acid, Sucrose, Threitol, Threonic acid, Threonine, Thymine, Tocopherol alpha-, Tryptophan, Tyrosine, 3-hydroxybutyric acid, Uracil, Urea, Uric acid, Valine, Xanthine, Xylitol, 3-phenyllactic acid, 3-phosphoglycerate, 4-aminobutyric acid, 4-hydroxybutyric acid, 4-hydroxyphenylacetic acid, 4716, 5-aminovaleric acid, 1-monopalmitin, Acetophenone, Adenine, Adenosine-5-monophosphate, Alanine, Alanine-alanine, Aminomalonate, Arabitol, Arachidic acid, Asparagine, Benzoic acid, 1-monostearin, Beta-glycerolphosphate, Butane-2,3-diol, Butylamine, Butyrolactam, Cellobiose, Cerotinic acid, Cholesterol, Citramalic acid, Citric acid, Citrulline, 1,5-anhydroglucitol, Creatinine, Cysteine, Cystine, D-erythro-sphingosine, Dodecanol, Fructose, Fructose-6-phosphate, Fucose, Fumaric acid, Galactinol, 2-aminobutyric acid, Glucose, Glucose-1-phosphate, Glucose-6-phosphate, Glutamic acid, Glutamine, Glutaric acid, Glyceric acid, Glycerol, Glycerol-alpha-phosphate, Glycine, 2-deoxypentitol, Glycolic acid, Glycyl tyrosine, Glycyl-proline, Guanidinosuccinate, Guanine, Heptadecanoic acid, Homoserine, Indole-3-acetate, Inulotriose, Isoleucine, 2-hydroxyglutaric acid, Isomaltose, Isothreonic acid, Lactic acid, Lauric acid, Leucine, Levoglucosan, Lysine, Maleic acid, Maleimide, Malonic acid, 2-ketoisocaproic acid, Maltose, Maltotriose, Melezitose, Methionine, Methionine sulfoxide, Myo-inositol, Myristic acid, N-carbamoylaspartate, Nicotinic acid, Octadecanol, 2,5-dihydroxypyrazine, Oleamide, Oleic acid, Ornithine, Oxoproline, Palmitic acid, Phenylacetic acid, Phenylalanine, Phosphate, Phosphoethanolamine, and Piperidone.

20. The method of claim 19, wherein the salivary biomarkers of HCC are selected from the group consisting of octadecanol, acetophenone, lauric acid, 1-monopalmitin, dodecanol, salicylaldehyde, glycyl-proline, 1-monosterin, creatinine, glutamine, serine, and 4-hydroxybutyric acid.

21. The method of claim 20, wherein the salivary biomarkers of HCC are selected from the group consisting of acetophenone, octadecanol, lauric acid, 3-hydroxybutyric acid, 1-monopalmitin, 1-monostearin and combinations thereof.
